(19) United States
(12) Patent Application Publication
(10) Pub. No.: US 2022/0186228 A1
(43) Pub. Date: Jun. 16, 2022

(54) SYNTHETIC MICRORNA MIMICS

(71) Applicant: RNATIVES INC., Boston, MA (US)

(72) Inventors: Mikko Petri TURUNEN, Kuopio (FI); Tiia Annika TURUNEN, Kuopio (FI); Seppo YLÄ-HERTTUALA, Kuopio (FI); Pia LAITINEN, Kuopio (FI); Mari-Anna VÄÄNÄNEN, Kuopio (FI)

(73) Assignee: RNATIVES INC., Boston, MA (US)

(21) Appl. No.: 17/415,155

(22) PCT Filed: Dec. 19, 2019
(86) PCT No.: PCT/FI2019/050906

§ 371 Date: Jun. 17, 2021

(30) Foreign Application Priority Data
Dec. 20, 2018 (FI) 20186118

(51) Int. Cl. C12N 15/113; G01N 33/68

(52) U.S. Cl. CPC C12N 15/1136; C12N 2310/141; G01N 33/6893

(57) ABSTRACT

The present invention relates to the treatment of cardiovascular diseases. In particular the present invention relates to micro RNA (mi RNA) molecules for use in the regulation of the gene expression of Vascular endothelial growth factor A (VEGFA), Vascular endothelial growth factor D (VEGFD) and/or Hypoxia-inducible factor 1-alpha (HIF1A) in a variety of applications, including use in therapeutic and diagnostic applications. VEGFA has diverse functions in both developing and mature individuals. VEGFA is a well-known critical regulator of angiogenesis and is also involved in the development and metastasizing of cancer.

### CROSS-REFERENCE TO RELATED APPLICATIONS

This application is a 35 U.S.C. § 371 National Phase Entry Application of International Application No. PCT/F12019/050906 filed Dec. 19, 2019, which claims benefit under 35 U.S.C. § 119(a) of Fl Application No. 20186118 filed Dec. 20, 2018, the contents of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jun. 25, 2020, is named P2318PC00_seqlisting.txt and is 28,296 bytes in size.

### FIELD OF THE DISCLOSURE

The present invention relates to the treatment of cardiovascular diseases. In particular, the present invention relates to microRNA (miRNA) molecules for use in the regulation of the gene expression of Vascular endothelial growth factor A (VEGFA), Vascular endothelial growth factor D (VEGFD) and/or Hypoxia-inducible factor 1-alpha (HIF1A) in a variety of applications, including use in therapeutic and diagnostic applications. VEGFA has diverse functions in both developing and mature individuals. VEGFA is a well-known critical regulator of angiogenesis and is also involved in the development and metastasizing of cancer.

### BACKGROUND OF THE DISCLOSURE

Cardiovascular diseases (CVDs) are one of the top causes of death globally, and it is predicted that the number of deaths will increase in the future due to population aging and a prolonged average life expectancy (Top 10 causes of death, WHO, 2017). CVDs include a broad group of illnesses involving the heart and the blood vessels, so the underlying pathological mechanism varies depending on the disease in question. However, many cardiovascular diseases share a common underlying feature that is the gradual or acute blood vessel occlusion. Once a blood vessel is occluded, the oxygen supply to the tissues is compromised and tissues have to adapt to hypoxic conditions on time in order to survive. However, this adaption process involves the growth of new co-lateral blood vessels by angiogenesis, which takes a long physiological time.

The angiogenesis is a highly regulated process and although well-studied, there is not yet a full understanding of its regulatory mechanisms. All living cells adapt to new environmental conditions like hypoxia by transcribing the right set of genes that will ensure its survival. Gene expression is a highly controlled event and regulatory mechanisms are exerted at transcriptional and post-transcriptional levels. In the case of lack of oxygen supply, one of the most important upregulated genes in the cell is the VEGFA. This gene has been reported to have a highly intricate regulation. When VEGFA is upregulated in tissues under hypoxic conditions it promotes angiogenesis, vasculogenesis and endothelial cell growth. It is known that VEGFA protein is secreted out of the cells and has paracrine and autocrine effects acting on two different receptors, VEGFR1 and

VEGFR2, which mediate its action. Imbalances in the regulation of VEGFA gene expression contribute to ischemic or malignant disorders, and it is involved in both the physiological and pathological angiogenesis. For example, in cancer, VEGFA upregulation has been correlated with a bad prognosis. There is a need to elucidate the transcriptional regulatory mechanisms of VEGFA in order to develop new therapies that would allow local modulation of angiogenesis.

It is known that VEGFA is regulated on many different levels transcriptionally and post-transcriptionally: Firstly, alternative splicing of different transcript variants generates diverse isoforms. Secondly, at post-transcriptional level, VEGFA messenger RNA (mRNA) stability is regulated by effectors such as hypoxia through the binding of stabilizing and destabilizing proteins. Thirdly, it has been recently discovered that VEGFA mRNA 3′ untranslated region (3′ UTR) works as a protein-dependent riboswitch undergoing a conformational change after binding proteins that control its stability, instead of the classical metabolite-interacting riboswitches. Fourth, the translation of VEGFA mRNA is highly controlled by miRNAs targeting of the 3′UTR by base-pairing mechanism and inducing gene silencing. These different levels of regulation cooperate in the fine-tuning of the expression of VEGFA variants.

VEGFD is another member of the VEGF/PDGF family of structurally related proteins and is a potent angiogenic cytokine. It promotes endothelial cell growth, promotes lymphangiogenesis, and can also affect vascular permeability.

HIF1A, also known as HIF-1-alpha, is a subunit of a heterodimeric transcription factor hypoxia-inducible factor 1 (HIF-1) that is encoded by the HIF1A gene. It is considered as the master transcriptional regulator of cellular and developmental response to hypoxia. The dysregulation and overexpression of HIF1A by either hypoxia or genetic alternations have been heavily implicated in cancer biology, as well as a number of other pathophysiologies, specifically in areas of vascularization and angiogenesis, energy metabolism, cell survival, and tumor invasion.

MicroRNAs (miRNAs, miRs) are endogenous non-coding, typically 19-22 nucleotide, RNAs that affect the protein expression of their target genes by transcript cleavage or translation blockade. miRNAs are a class of genome-encoded small RNAs, mostly encoded within intronic or exonic regions of protein-coding genes (host genes). Increasing evidence suggests a functional relationship between these miRNAs and their host genes, so that when the host gene is up- or downregulated, its encoded miRNAs follow the same trend. Moreover, miRNAs have a complex biogenesis: first, they are transcribed by RNA polymerase II (pol II) to generate the primary transcripts (pri-miRNAs). Then, the highly conserved Drosha protein, in complex with DGCR8, processes the pri-miRNAs in the nucleus to generate pre-miRNA hairpins. Pre-miRNAs are exported to the cell cytoplasm through nuclear pore complexes by RanGTP•exporting•cargo complexes. The exportation to the cytoplasm establishes a compartmentalization between two processing steps of the miRNAs, the one performed by Drosha in the nucleus and a second one performed by Dicer enzyme in the cytoplasm. Following nuclear export, the cytoplasmic RNAse III Dicer participates in the second processing step (‘dicing’) to produce miRNA duplexes of ˜22-nucleotides in length. Dicer is a highly conserved protein and it is found in almost all eukaryotic organisms. The Dicer cleavage product, the miRNA double-stranded duplex-structure, is composed by a miRNA-5p strand and a −3p strand and does not persist in the cell for long. The 5p strand is present in the forward (5′-3′) position and 3p strand (which is almost complimentary to the 5p strand) is in the reverse position. Usually, the miRNA duplex is incorporated into the RISC complex (formed by Dicer, TRBP and Ago2 proteins). RISC complex recognizes the miRNA, unwinds it and selects the guide miRNA strand (guide strand) while degrading the passenger strand.

The ‘strand bias’ selection theory was proposed after analysing the thermodynamic stability profiles of pre-miRNAs and mature miRNAs. However, recent data suggest that the strand selection on miRNAs might be tissue dependent. Moreover, it has been reported that a significant fraction of miRNA sister strand pairs is expressed at comparable level showing a not deterministic strand selection bias. The two mature miRNAs from the same hairpin precursor seem to have different sets of target genes and may differently contribute to the regulation of cell activities.

The mature miRNAs are primarily considered to be post-transcriptional negative regulators, targeting the 3′UTR region of mRNAs in the cytoplasm by sequence complementarity. The canonical view of miRNA post-transcriptional gene regulatory function is that miRNA binding to the target mRNA inhibits the translation of mRNA into protein. The region of the miRNA with high complementarity to its target mRNA consists of 2-8 nucleotides at the 5′ end and it is known as the “seed” region.

However, it is becoming increasingly evident that this canonical view of miRNAs inducing post-transcriptional gene silencing is incomplete, and miRNAs might have additional effects. One reason is the fact that the majority of cellular mature miRNAs are present in both the nucleus and the cytoplasm, and some miRNAs even show specific nuclear enrichment like the miR-21 or miR-29b.

Increasing evidence points to gene regulatory functions for miRNAs in the nucleus of the mammalian cells. It has been demonstrated that small RNAs (sRNAs), such as short hairpin RNAs (shRNAs) and small interfering RNAs (siRNAs), can actually induce epigenetic alterations that either silence or activate transcription at specific gene promoters. There is evidence demonstrating that sRNA-regulated gene expression is mediated by sequence complementarity between the sRNA and the promoter sequence. In addition, sRNAs have been reported to modulate co-transcriptional alternative splicing events.

Although available data detail the protein effectors of this sRNA-guided transcriptional control, the sequence recognition mechanism by which sRNAs, such as miRNAs, identify their nuclear targets remains unclear. One possible explanation for the sRNA genome-sequence recognition could lie in the observation of a bi-directional transcription in the human genome. Human genome is transcribed in both sense and antisense directions, originating non-coding sense and anti-sense transcripts located in the promoter region of many genes. It is believed that miRNAs may act at transcriptional level by three mechanisms: i) interacting with the above mentioned promoter associated non-coding transcripts; ii) interacting with single stranded DNA, forming DNA-RNA hybrids; iii) interacting with double-stranded DNA, forming RNA*DNA:DNA triplexes.

Document KR20150095349 discloses a pharmaceutical composition for preventing or treating nervous system disorders through control of microRNA. Also disclosed are a marker usable as a diagnosis or a prognosis of nervous system disorders and uses thereof. Document WO2016170348 A2 discloses oligonucleotides, e.g., small activating RNAs (saRNAs) useful in upregulating the expression of a target gene and therapeutic compositions comprising such oligonucleotides. Methods of using the oligonucleotides and the therapeutic compositions are also provided. Document WO2015023975 A1 relates to methods for increasing gene expression in a targeted manner. In some embodiments, methods and compositions are provided that are useful for post-transcriptionally altering protein and/or RNA levels in a targeted manner. Document also provides methods and compositions that are useful for protecting RNAs from degradation (e.g., exonuclease mediated degradation).

The tissue hypoxia and subsequent angiogenesis can be understood from two opposite perspectives in cardiovascular diseases and cancer. The growth of new blood vessels can guarantee the tissue survival in certain situations. However, it can facilitate the growth and migration of malignant cells in tumours. Regulating angiogenesis and being able to promote or inhibit it locally could be used e.g. as a therapy for treating cardiovascular diseases that involve limited blood oxygen supply. Many diseases, such as myocardial infarction or peripheral ischemia, could be treated by modulating the amount of angiogenic factors like VEGFA. There are several methods for downregulation of VEGFA (eg. antibodies, siRNAs) but currently the only method for upregulation of VEGFA is traditional gene delivery using different vectors (modified viruses, non-viral vectors, etc.). Traditional gene delivery suffers from several drawbacks: efficiency of delivery is usually weak, vectors and transgenes can cause immunological reactions, typically only one isoform of a gene is delivered and natural balance on different isoforms is unbalanced, and vectors can integrate their transgene in random sites at chromatin: possibly causing oncogenesis. However, despite extensive efforts to develop VEGFA therapies, a uniformly effective VEGFA treatment has not been developed.

### BRIEF DESCRIPTION OF THE DISCLOSURE

It is thus known that miRNAs act canonically at post-transcriptional level targeting the 3′UTR of mRNAs, but a novel regulation mechanism suggests that miRNAs can interact with gene promoters they share sequence similarity with. The present inventors investigated the functions of a murine miRNA (mmu-miR-466c or mmu-miR-466c-1), that is upregulated in hypoxia and has putative binding sites within the Vegfa promoter. miRNA-466c belongs to miRNA-Family MIPF0000208 that comprises also the following miRNAs: mmu-miR-466a, mmu-miR-466b-1, mmu-miR-466b-2, mmu-miR-466b-3, mmu-miR-466e, mmu-miR-466f-4, mmu-miR-466k, mmu-miR-466c-2, mmu-miR-466b-4, mmu-miR-466b-5, mmu-miR-466b-6, mmu-miR-466b-7, mmu-miR-466p, mmu-miR-466b-8 and mmu-miR-466c-3.

The results obtained in the present study by transfection of a hairpin mimic of miR-466c (miR-466c 3p mim), a single stranded 3p mimic (ssRNA-466c-3p) and a duplex structure of this miRNA, which include the 3p strand and 5p strand annealed, suggest that there may exist a strand bias selection in human endothelial cells. Moreover, the present inventors were able to see clear differences in VEGFA regulation when different types of mimics were used in transfections. The present inventors show for the first time ever that hairpin mimics miR-466c-3p and miR-466c-5p mim are able to strongly upregulate VEGFA, whereas the rest of mimics induce only slight changes or no changes in VEGFA mRNA levels.

The present inventors thus demonstrate that synthetic mimics of mmu-miR-466c are able to regulate the mRNA levels of human VEGFA, although in a different manner as it was observed previously in mouse Vegfa mRNA. Based on a bioinformatic analysis done in order to identify promoters targeted by miR-466c, similar regulation mechanism by miR-466c also exists for VEGFD and HIFA mRNAs. The present inventors found that two microRNAs—hsa-miR-297 and hsa-miR-574-3p—share an especially high amount of sequence similarity with mmu-miR-466c. Due to the high similarity shared between the mouse miRNAs and human miRNAs, it is reasonable to assume that the human miRNA counterparts have similar targets and that mouse miRNAs transfected into human cells are able regulate human VEGFA, VEGFD and HIF1A mRNA levels.

The benefits of the present invention are that large amounts of miRNAs can be produced by lentivirus. RNAs can be also synthetic (eg. RNA-oligos, unmodified or chemically modified) which can be delivered to target cells by physical or chemical methods. In addition, these RNAs can be delivered to target cells and tissues by extracellular vesicles, which are naturally existing nanoparticles that carry small RNAs. Exosomes can be loaded with RNAs by various methods, including RNA expression in producer cells and RNA loading by sonication or electroporation.

An object of the present disclosure is to provide a synthetic miRNA molecule having at least 80% sequence identity to SEQ ID NO: 5, SEQ ID NO: 6, SEQ ID NO: 3, SEQ ID NO: 4, SEQ ID NO: 15 or SEQ ID NO: 16 or a complementary sequence thereof. Another object of the invention is to provide a pharmaceutical composition comprising at least one synthetic miRNA molecule having at least 80% sequence identity to SEQ ID NO: 5, SEQ ID NO: 6, SEQ ID NO: 3, SEQ ID NO: 4, SEQ ID NO: 15 or SEQ ID NO: 16 or a complementary sequence thereof and a carrier or vehicle. A recombinant expression vector comprising at least one synthetic miRNA molecule of the invention is also an aspect of the invention. Also, a cell comprising said recombinant expression vector is an aspect of the invention.

A method of modulating the expression of a target protein in human, comprising administering the synthetic miRNA molecule having at least 80% sequence identity to SEQ ID NO: 5, SEQ ID NO: 6, SEQ ID NO: 3, SEQ ID NO: 4, SEQ ID NO: 15 or SEQ ID NO: 16 or a complementary sequence thereof is also an aspect of the invention.

Another aspect of the invention is a method for determining whether a subject is at risk of or suffering from a cardiovascular disease, the method comprising the steps of:

(A) providing a sample of an individual who is suspected of cardiovascular disease;

(B) optionally extracting RNA from said sample;

(C) measuring the expression level of hsa-miR-297 or hsa-547-3p by qRT-PCR; wherein a decreased level of expression of hsa-miR-297 or hsa-547-3p compared to a control sample indicates a presence of or a predisposition to cardiovascular disease.

### BRIEF DESCRIPTION OF THE DRAWINGS

FIG. 1 shows a graphic genome location of mmu-miR-466c that is encoded in intron 10 region of the Sfmbt2 gene.

FIG. 2 shows shRNA and mmu-miR-466c target sites on mouse Vegfa promoter. Schematic illustration shows the proximity between the target sites of shRNA-451 used previously for transcriptional gene activation and the predicted target site of mmu-miR-466c on the murine Vegfa promoter.

FIG. 3 shows alignment of mmu-miR-466c-5p sequence with hsa-miR-574-3p and hsa-miR-297 sequences.

FIG. 4 illustrates that removal of mmu-miR-466c abolishes the induction of Vegfa in response to hypoxia. A. Hypoxia induces mmu-miR-466c expression in mouse endothelial cells (C166). After removing the mmu-miR-466c from the cell genome using CRISPR, this upregulation was not observed. B. Removal of mmu-miR-466 reduces the expression of Sfmbt2 mRNA. C. Vegfa was not upregulated upon hypoxia when mmu-miR-466c was removed from the genome. D. Lentiviral transduction of mmu-miR-466c (LV-466) increases Vegfa expression but does not rescue Vegfa induction in hypoxia. qRT-PCR results calculated relative to normoxia and normalized to Actb expression. n=3, mean+SD.

FIG. 5 Transfection of murine miR-466c hairpin mimic miR-466c-3p mim significantly decreased VEGFA mRNA levels in human cell line Ea.hy926, whereas the transfection of the miR-466c-5p mimic had no significant effect when compared to negative control transfection. qPCR results were calculated relative to negative control samples by log 2 fold change. Results are expressed n=3, mean±SD. Results normalized to ACTB expression. P-value *<0.05.

FIG. 6 shows that the transfection of murine single-stranded miR-466c-3p mimics (ssRNA-466 3p) and duplex mimic (466c duplex) significantly decreased VEGFA mRNA levels in human cell line ARPE-19, when compared to negative control transfection. qPCR results were calculated relative to negative control samples by log 2 fold change. Results are expressed n=3, mean±SD. Results normalized to ACTB expression. P-value *<0.05.

FIG. 7 shows that VEGFA expression is increased in ARPE-19 cells 5 days after transduction with LV-mmu-miR-466. Results are expressed n=3, mean±SD. Results normalized to ACTB expression. P-value *<0.05.

FIG. 8 shows that transfection of murine miR-466c hairpin mimic miR-466c-3p mim and miR-466c-5p mimic significantly increase VEGFA mRNA levels in human cell line ARPE-19. qPCR results were calculated relative to negative control samples by log 2 fold change. Results are expressed n=3, mean±SD. Results normalized to ACTB expression. P-value *<0.05.

FIG. 9 shows that synthetic 5p fusion and 5p repeat RNA, containing seed sequence of miR-466c-5p and either scrambled nucleotides (5p fusion) or dinucleotide repeat (5p repeat), both increase VEGFA expression 2-fold in ARPE-19 cells. Results are expressed n=6, mean±SD. Results normalized to ACTB expression.

FIG. 10 illustrates that transfection of murine single-stranded miR-466c-3p (ssRNA-466 3p) and duplex mimic (466c duplex) do not change VEGFA mRNA levels in human ARPE-19 cells, whereas the transfection of single-stranded miR-466c-5p induced an upregulation of VEGFA, when compared to negative control transfection. qPCR results were calculated relative to negative control samples by log 2 fold change. Results are expressed n=3, mean±SD. Results normalized to ACTB expression. P-value *<0.05.

FIG. 11 presents a hypothesis of the pathway of mmu-miR-466 controlling Vegfa transcription. mmu-miR-466c is encoded in the intron 10 of the Sfmbt2 gene. miRNA is transcribed and processed in the canonical pathway of miRNAs maturation by Drosha and Dicer enzyme complexes. The mature form of miRNA may regulate Vegfa transcription by interacting with its promoter region. This interaction might induce epigenetic changes resulting in transcriptional gene silencing (TGS) or activation (TGA). It is also possible that the mature form of the miRNA acts at cytoplasmic level targeting the 3′UTR region of Vegfa mRNA and, inducing post-transcriptional gene silencing (PTGS).

FIG. 12 shows a plasmid map for Cas9 from S. pyogenes with 2A-EGFP, and cloning backbone for sgRNA [p5p-Cas9(BB)-2A-GFP (PX458) (#48138, Addgene)].

FIG. 13 illustrates cell viability after miRNA administration.

FIG. 14 is a schematic figure of miRNA packaging in extracellular vesicles.

FIG. 15 presents the results of fusion protein expression: a) RNA binding domain (Ago2), b) EV-specific transmembrane protein domain (CD9), c) DAPI and d) merge of signals of RNA binding domain (Ago2) and EV-specific transmembrane protein domain (CD9).

FIG. 16 shows the presence of EV-loaded miRNA in recipient cells.

### SEQUENCE LISTING

SEQ ID NO: 1 Mouse VEGFA promoter nucleotide sequence SEQ ID NO: 2 Human VEGFA promoter nucleotide sequence

SEQ ID NO: 3 mmu-miR-466c-5p (MIMAT0004877) SEQ ID NO: 4 mmu-miR-466c-3p (MIMAT0004878)

SEQ ID NO: 5 Synthetic RNA comprising custom GU-repeat throughout the sequence (Repeat-miR-466c-5p)

SEQ ID NO: 6 Synthetic RNA that has GU-repeat containing seed combined with scrambled sequence with no existing RNA target (custom fusion; fusion-miR466c-5p)

SEQ ID NO: 7 Cloning oligos of mmu-miR-466c for lentiviral vector overexpression: Forward cloning primer

SEQ ID NO: 8 Cloning oligos of mmu-miR-466c for lentiviral vector overexpression: Reverse cloning primer

SEQ ID NO: 9 CRISPR mmu-miR-466c deletion guide oligos: M-miR-466 1a miR-466 1 gRNA T

SEQ ID NO: 10 CRISPR mmu-miR-466c deletion guide oligos: M-miR-466 1 b miR-466 1 gRNA B

SEQ ID NO: 11 CRISPR mmu-miR-466c deletion guide oligos: M-miR-466 2a miR-466 2 gRNA T

SEQ ID NO: 12 CRISPR mmu-miR-466c deletion guide oligos: M-miR-466 2b miR-466 2 gRNA B

SEQ ID NO: 13 CRISPR deletion detection PCR primers: 466del forward primer

SEQ ID NO: 14 CRISPR deletion detection PCR primers: 466del reverse primer

SEQ ID NO: 15 hsa-miR-297 sequence (MIMAT0004450)

SEQ ID NO: 16 hsa-miR-574-3p sequence (MIMAT0003239)

SEQ ID NO: 17 Synthetic RNA comprising only GU-repeat throughout the sequence (Repeat only miR-466c)

SEQ ID NO: 18 Synthetic RNA comprising custom GU-repeat seed and miR-466c-5p sequence (Seed repeat miR-466c-5p)

SEQ ID NO: 19 Synthetic RNA comprising custom scrambled sequence with no existing RNA target seed and miR-466c-5p sequence (Seed-fusion miR-466c-5p)

SEQ ID NO: 20 hCD9 forward primer (with Kozak, without stop codon) related to cloning of human LV.CD9.hAgo2

SEQ ID NO: 21 hCD9 reverse primer (with Kozak, without stop codon) related to cloning of human LV.CD9.hAgo2

SEQ ID NO: 22 hCD9 forward primer (15 bp homology with LV.vector) related to cloning of human LV.CD9.hAgo2

SEQ ID NO: 23 hCD9 reverse primer (15 bp homology with LV.vector) related to cloning of human LV.CD9.hAgo2

SEQ ID NO: 24 Forward primer for HA.FLAG.hAgo2 related to cloning of human LV.CD9.hAgo2

SEQ ID NO: 25 Reverse primer for HA.FLAG.hAgo2 related to cloning of human LV.CD9.hAgo2

SEQ ID NO: 26 hTSG101 forward primer (with Kozak, without stop codon) related to cloning of hTSG101.hAgo2

SEQ ID NO: 27 hTSG101 reverse primer (with Kozak, without stop codon) related to cloning of hTSG101.hAgo2

SEQ ID NO: 28 hTSG101 forward primer (20 bp homology with LV.vector) related to cloning of hTSG101.hAgo2

SEQ ID NO: 29 hTSG101 reverse primer (20 bp homology with LV.vector) related to cloning of hTSG101.hAgo2

SEQ ID NO: 30 Forward primer for HA.FLAG.hAgo2 related to cloning of hTSG101.hAgo2

SEQ ID NO: 31 Reverse primer for HA.FLAG.hAgo2 related to cloning of hTSG101.hAgo2

SEQ ID NO: 32 LV.CD9.Ago2 cDNA as cloned

SEQ ID NO: 33 hTSG101.Ago2 cDNA as cloned

### DETAILED DESCRIPTION OF THE DISCLOSURE

The term “miRNA” is used according to its usual meaning and current and refers to a microRNA molecule found in eukaryotes which is involved in gene regulation based on RNA. The term “miRNA,” unless otherwise indicated, refers to the processed RNA, after it has been cleaved from its precursor. The term “miRNA” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary. The miRNAs according to the present invention include mmu-miR-466c family MIPF0000208 members i.e. mmu-miR-466a, mmu-miR-466b-1, mmu-miR-466b-2, mmu-miR-466b-3, mmu-miR-466c-1, mmu-miR-466e, mmu-miR-466f-4, mmu-miR-466k, mmu-miR-466c-2, mmu-miR-466b-4, mmu-miR-466b-5, mmu-miR-466b-6, mmu-miR-466b-7, mmu-miR-466p, mmu-miR-466b-8 and mmu-miR-466c-3. In addition, miRNAs according to the invention include hsa-miR-297 and hsa-miR574-3p.

The term “complementary to 5′” in the context means being able to hybridize with the target antisense RNA transcript under stringent conditions. The term “antisense” when used to describe a target antisense RNA transcript in the context of the present invention means that the sequence is complementary to a sequence on the coding strand of a gene. It is to be understood that thymidine of the DNA is replaced by uridine in RNA and that this difference does not alter the understanding of the terms “antisense” or “complementarity”.

miRNAs regulate the gene expression by binding to the mRNA. The “seed sequence” or “seed region” in the context is a conserved heptametrical sequence which is mostly situated at positions 2-8 from the miRNA 5″-end and is essential for the binding of the miRNA to the mRNA. Even though base pairing of miRNA and its target mRNA does not match perfect, the “seed sequence” has to be perfectly complementary. The seed sequence according to the present invention can be such as mmu-miR-466c-5p seed sequence GAUGUGU or miR-466c-3p seed sequence UACAUAC or that of hsa-miR-574-3p UGUAUGUG.

The “coding strand” of a gene has the same base sequence as the mRNA produced, except T is replayed by U in the mRNA. The “template strand” of a gene is therefore complementary and antiparallel to the mRNA produced.

The term “transcription start site” (TSS) as used herein means a nucleotide on the template strand of a gene corresponding to or marking the location of the start of transcription. The TSS may be located within the promoter region on the template strand of the gene.

The term “transcription stop site” as used herein means a region, which can be one or more nucleotides, on the template strand of a gene, which has at least one feature such as, but not limited to, a region which encodes at least one stop codon of the target transcript, a region encoding a sequence preceding the 3′UTR of the target transcript, a region where the RNA polymerase releases the gene, a region encoding a splice site or an area before a splice site and a region on the template strand where transcription of the target transcript terminates.

As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules. In the present invention, miRNA can act by recruiting transcription factors to the target gene promoter. Examples of recruited transcription factors are chromatin modulating transcription factors and also other transcription factors, such as CREB2/ATF4, p300, YY1, GATA4, CRTC, SUZ12, POU2F7, EZH2 and Sp1.

The term “protein expression” refers to the process by which a nucleic acid sequence undergoes translation such that detectable levels of the amino acid sequence or protein are expressed.

As used herein, “reducing”, “suppression”, “silencing”, and “inhibition” are used interchangeably to denote the down-regulation of the level of expression of a product of a target sequence relative to its normal expression level in a wild type organism. By “reducing the level of RNA” or “reducing the amount of protein” is intended a reduction in expression by any statistically significant amount including, for example, a reduction of at least 10%, 15%, 20%, 25%, 30%, 35%, 40%, 45%, 50%, 55%, 60%, 65%, 70%, 75%, 80%, 85%, 90%, 95%, or 100% relative to the wild type expression level.

As used herein, “increasing” and “upregulating” are used interchangeably to denote the up-regulation of the level of expression of a product of a target sequence relative to its normal expression level in a wild type organism. By “increasing the level of RNA” or “increasing the amount of protein” is intended an increase in expression by any statistically significant amount including, for example, an increase of at least 10%, 15%, 20%, 25%, 30%, 35%, 40%, 45%, 50%, 55%, 60%, 65%, 70%, 75%, 80%, 85%, 90%, 95%, or 100% relative to the wild type expression level.

The term “recombinant” generally refers to a molecule that has been manipulated in vitro or that is the replicated or expressed product of such a molecule.

RNA molecules may be encoded by a nucleic acid molecule comprised in a vector. Generally, “operably linked” means that the DNA sequences being linked are contiguous, although they need not be, and that a gene and a regulatory sequence or sequences (e.g., a promoter) are connected in such a way as to permit gene expression when the appropriate molecules (e.g., transcriptional activator proteins “transcription factors” or proteins which include transcriptional activator domains) are bound to the regulatory sequence or sequences.

As used herein, the term “vector” refers to a nucleic acid construct designed for transduction/transfection of one or more cell types. Vectors may be, for example, “cloning vectors”, which are designed for isolation, propagation and replication of inserted nucleotides, “expression vectors”, which are designed for expression of a nucleotide sequence in a host cell, a “viral vector”, which is designed to result in the production of a recombinant virus or virus-like particle, or “shuttle vectors”, which comprise the attributes of more than one type of vector. The term “replication” means duplication of a vector.

The term “expression vector” is used interchangeably herein with the term “plasmid” and “vector” and refers to a recombinant DNA molecule containing a desired coding sequence and appropriate nucleic acid sequences necessary for expression of the operably linked coding sequence (e.g., an insert sequence that codes for a product) in a particular host cell. The term “plasmid” refers to an extrachromosomal circular DNA capable of autonomous replication in a given cell. In certain embodiments, the plasmid is designed for amplification and expression in bacteria.

Thus, embodiments provide nucleic acid constructs in the form of plasmids, vectors, transcription or expression cassettes which comprise at least one nucleotide sequence encoding a miRNA described herein, or fragments thereof, and a suitable promoter region. Suitable vectors can be chosen or constructed, which contain appropriate regulatory sequences, such as promoter sequences, terminator sequences, polyadenylation sequences, enhancer sequences, marker genes and other sequences as desired. Vectors can be plasmids, extracellular vesicles (either natural or modified), phage (e.g. phage, or phagemid) or viral (e.g. lentivirus, adenovirus, AAV) or any other appropriate vector. Non-limiting examples of vectors include plasmids, viral vectors (e.g., derived from lentivirus, adenovirus, adeno-associated virus (AAV), retrovirus, etc.), bacteriophage, cosmids, and artificial chromosomes. In embodiments, the vector can be an expression (or expression constructs) for driving expression of the polynucleotide in a target cell. Vectors and methods for inserting them into a target cell are known in the art [See, e.g., Sambrook et al., 1989].

Examples of seed-preferred promoters include, but are not limited to VEGFA promoter, VEGFD promoter and HIF1A promoter.

In one embodiment, the DNA molecule is operably linked to a recombinant expression vector. In a further embodiment, the vector is a lentiviral vector. In one embodiment, the presently disclosed construct is in a lentiviral backbone, i.e., a lentiviral vector, making production of active lentiviral particles possible, therefore also allowing effective expression even in cells having low transfection efficiency. In other embodiments, the presently disclosed synthetic mmu-miR-466c is cloned into other vectors that allow expression in cells, such as a lentivirus based vector and the like.

As used herein a “derivative” refers to a chemically modified or altered form of a naturally occurring molecule, while the terms “mimic” or “analog” refer to a molecule that may or may not structurally resemble a naturally occurring molecule or moiety but possesses similar functions. As used herein, a “moiety” generally refers to a smaller chemical or molecular component of a larger chemical or molecular structure. Nucleobase, nucleoside and nucleotide analogs or derivatives are well known in the art. The most preferred aspect of the invention is an miRNA mimic comprising a seed sequence of mmu-miR-466c-5p GAUGUGU and mmu-miR-466c-3p UACAUAC. In a further aspect the miRNA is mmu-miR-466c-5p or an miRNA mimic comprising the mature sequence thereof.

The term “short” refers to a length of a single polynucleotide that is 150 nucleotides or fewer. The nucleic acid molecules are synthetic. The term “synthetic” means the nucleic acid molecule is isolated and not identical in sequence (the entire sequence) and/or chemical structure to a naturally-occurring nucleic acid molecule, such as an endogenous precursor miRNA molecule. While in some embodiments, nucleic acids of the invention do not have an entire sequence that is identical to a sequence of a naturally-occurring nucleic acid, such molecules may encompass all or part of a naturally-occurring sequence. It is contemplated, however, that a synthetic nucleic acid administered to a cell may subsequently be modified or altered in the cell such that its structure or sequence is the same as non-synthetic or naturally occurring nucleic acid, such as a mature miRNA sequence. For example, a synthetic nucleic acid may have a sequence that differs from the sequence of a precursor miRNA, but that sequence may be altered once in a cell to be the same as an endogenous, processed miRNA. It is understood that a “synthetic nucleic acid” of the invention means that the nucleic acid does not have a chemical structure or sequence of a naturally occurring nucleic acid. Consequently, it will be understood that the term “synthetic miRNA” refers to a “synthetic nucleic acid” that functions in a cell or under physiological conditions as a naturally occurring miRNA.

In particular embodiments, the nucleic acid in methods and/or compositions of the invention is specifically a synthetic miRNA and not a non-synthetic miRNA (that is, not an miRNA that qualifies as “synthetic”); though in other embodiments, the invention specifically involves a non-synthetic miRNA and not a synthetic miRNA.

Any embodiments discussed with respect to the use of synthetic miRNAs can be applied with respect to non-synthetic miRNAs, and vice versa.

The synthetic polynucleotides of the invention include untranslated regions. Untranslated regions (UTRs) of a gene are transcribed but not translated. The 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation.

The term “isolated” means that the nucleic acid molecules of the invention are initially separated from different (in terms of sequence or structure) and unwanted nucleic acid molecules such that a population of isolated nucleic acids is at least about 90% homogenous, and may be at least about 95, 96, 97, 98, 99, or 100% homogenous with respect to other polynucleotide molecules. In many embodiments of the invention, a nucleic acid is isolated by virtue of it having been synthesized in vitro separate from endogenous nucleic acids in a cell. It will be understood, however, that isolated nucleic acids may be subsequently mixed or pooled together.

As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another or have a certain amount of sequence identity to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

The term “targeting a miRNA to modulate” means a nucleic acid of the invention will be employed so as to modulate the selected miRNA. In some embodiments the modulation is achieved with a synthetic or non-synthetic miRNA that corresponds to the targeted miRNA, which effectively provides the targeted miRNA to the cell or organism (positive modulation). In other embodiments, the modulation is achieved with a miRNA inhibitor, which effectively inhibits the targeted miRNA in the cell or organism (negative modulation).

In certain embodiments, a synthetic miRNA has a nucleotide at its 5′ end of the complementary region in which the phosphate and/or hydroxyl group has been replaced with another chemical group (referred to as the “replacement design”). In some cases, the phosphate group is replaced, while in others, the hydroxyl group has been replaced. In particular embodiments, the replacement group is biotin, an amine group, a lower alkylamine group, an acetyl group, 2 O-Me (2 Oxygen-methyl), DMTO (4,4′-dimethoxytrityl with oxygen), fluorescein, a thiol, or acridine, though other replacement groups are well known to those of skill in the art and can be used as well. This design element can also be used with an miRNA inhibitor.

Additional embodiments concern a synthetic miRNA having one or more sugar modifications in the first or last 1 to 6 residues of the complementary region (referred to as the “sugar replacement design”). In certain cases, there is one or more sugar modifications in the first 1, 2, 3, 4, 5, 6 or more residues of the complementary region, or any range derivable therein. In additional cases, there is one or more sugar modifications in the last 1, 2, 3, 4, 5, 6 or more residues of the complementary region, or any range derivable therein, have a sugar modification. It will be understood that the terms “first” and “last” are with respect to the order of residues from the 5′ end to the 3′ end of the region. In particular embodiments, the sugar modification is a 2′O-Me modification. In further embodiments, there is one or more sugar modifications in the first or last 2 to 4 residues of the complementary region or the first or last 4 to 6 residues of the complementary region. This design element can also be used with an miRNA inhibitor. Thus, an miRNA inhibitor can have this design element and/or a replacement group on the nucleotide at the 5′ terminus, as discussed above.

In other embodiments of the invention, there is a synthetic miRNA in which one or more nucleotides in the last 1 to 5 residues at the 3′ end of the complementary region are not complementary to the corresponding nucleotides of the miRNA region (“noncomplementarity”) (referred to as the “noncomplementarity design”). The noncomplementarity may be in the last 1, 2, 3, 4, and/or 5 residues of the complementary miRNA. In certain embodiments, there is noncomplementarity with at least 2 nucleotides in the complementary region. It is contemplated that synthetic miRNA of the invention have one or more of the replacements, sugar modification, or noncomplementarity designs. In certain cases, synthetic RNA molecules have two of them, while in others these molecules have all three designs in place. The miRNA region and the complementary region may be on the same or separate polynucleotides. In cases in which they are contained on or in the same polynucleotide, the miRNA molecule will be considered a single polynucleotide. In embodiments in which the different regions are on separate polynucleotides, the synthetic miRNA will be considered to be comprised of two polynucleotides. When the RNA molecule is a single polynucleotide, there is a linker region between the miRNA region and the complementary region. In some embodiments, the single polynucleotide is capable of forming a hairpin loop structure as a result of bonding between the miRNA region and the complementary region. The linker constitutes the hairpin loop. It is contemplated that in some embodiments, the linker region is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 residues in length, or any range derivable therein, in certain embodiments, the linker is between 3 and 30 residues (inclusive) in length.

It will be understood that the term “naturally occurring” refers to something found in an organism without any intervention by a person; it could refer to a naturally-occurring wildtype or mutant molecule. In some embodiments a synthetic miRNA molecule does not have the sequence of a naturally occurring miRNA molecule. In other embodiments, a synthetic miRNA molecule may have the sequence of a naturally occurring miRNA molecule, but the chemical structure of the molecule, particularly in the part unrelated specifically to the precise sequence (non-sequence chemical structure) differs from chemical structure of the naturally occurring miRNA molecule with that sequence. In some cases, the synthetic miRNA has both a sequence and non-sequence chemical structure that are not found in a naturally-occurring miRNA. Moreover, the sequence of the synthetic molecules will identify which miRNA is effectively being provided or inhibited; the endogenous miRNA will be referred to as the “corresponding miRNA”. Corresponding miRNA sequences that can be used in the context of the invention include, but are not limited to, SEQ ID NO:3, SEQ ID NO: 4, SEQ ID NO:15 and SEQ ID NO:16.

In some embodiments, there is a synthetic miRNA having a length of between 17 and 130 residues. More preferably the length is 14 to 30 residues. The present invention concerns synthetic miRNA molecules that are, are at least, or are at most 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 residues in length, or any range derivable therein.

In certain embodiments, synthetic miRNA has a) an “miRNA region” whose sequence from 5′ to 3′ is identical to a mature miRNA sequence, and b) a “complementary region” whose sequence from 5′ to 3′ is between 60% and 100% complementary to the miRNA sequence. In certain embodiments, these synthetic miRNAs are also isolated, as defined above. The term “miRNA region” refers to a region on the synthetic miRNA that is at least 90% identical to the entire sequence of a mature, naturally occurring miRNA sequence. In certain embodiments, the miRNA region is or is at least 90, 91, 92, 93, 94, 95, 96, 97, 98, 99, 99.1, 99.2, 99.3, 99.4, 99.5, 99.6, 99.7, 99.8, 99.9 or 100% identical to the sequence of a naturally-occurring miRNA. The term “complementary region” refers to a region of a synthetic miRNA that is or is at least 60% complementary to the mature, naturally occurring miRNA sequence that the miRNA region is identical to. The complementary region is or is at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein. With single polynucleotide sequences, there is a hairpin loop structure as a result of chemical bonding between the miRNA region and the complementary region. In other embodiments, the complementary region is on a different nucleic acid molecule than the miRNA region, in which case the complementary region is on the complementary strand and the miRNA region is on the active strand.

In some embodiments, of the invention, a synthetic miRNA contains one or more design elements. These design elements include but are not limited to: i) a replacement group for the phosphate or hydroxyl of the nucleotide at the 5′ terminus of the complementary region; ii) one or more sugar modifications in the first or last 1 to 6 residues of the complementary region; or, iii) noncomplementarity between one or more nucleotides in the last 1 to 5 residues at the 3′ end of the complementary region and the corresponding nucleotides of the miRNA region.

It will be understood that the term “providing” an agent is used to include “administering” the agent to a patient.

The term “effective amount” as used herein is defined as the amount of the molecules of the present invention that are necessary to result in the desired physiological change in the cell or tissue to which it is administered. The term “therapeutically effective amount” as used herein is defined as the amount of the molecules of the present invention that achieves a desired effect with respect to a disease or condition. A skilled artisan readily recognizes that in many cases the molecules may not provide a cure but may provide a partial benefit, such as alleviation or improvement of at least one symptom. In some embodiments, a physiological change having some benefit is also considered therapeutically beneficial. Thus, in some embodiments, an amount of molecules that provides a physiological change is considered an “effective amount” or a “therapeutically effective amount.”

The term “pharmaceutically acceptable solvate”, as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-. and tri-hydrates), N-methylpyrrolidinone (NMP), dimethyl sulfoxide (DMSO), N,N′-dimethylformamide (DMF), N,N′-dimethylacetamide (DMAC), I,3-dimethyl-2-imidazolidmone (DMEU), I,3-dimethyl-3,4,5,6-tetrahydro-2-(IH)-pyrimidinone (DMPU), acetonitrile (ACN), propylene glycol, ethyl acetate, benzyl alcohol, 2-pyrrol idone. benzyl benzoate, and the like. When water is the solvent, the solvate is referred to as a “hydrate.”

As used herein, “pharmaceutically acceptable carrier” may include one or more solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Supplementary active ingredients also can be incorporated into the compositions.

Focusing on miRNA nuclear functions, Turunen et al. (Circ Res 105(6):604-609) previously found that small hairpin RNAs (shRNAs), synthetic counterparts of biological miRNAs, were able to activate or repress Vegfa expression when targeted to murine Vegfa gene promoter. The induced transcriptional gene activation (TGA) and transcriptional gene silencing (TGS) were dependent on the targeted sequence. The shRNA targeted to the Vegfa promoter resulted in the attraction of transcription factors and induced epigenetic changes both in vitro and in vivo.

The present inventors have also shown that shRNA-mediated transcriptional activation of the Vegfa promoter offers therapeutic improvement in murine models of hindlimb ischemia and myocardial infarction. These observations suggested that shRNAs may function by mimicking structurally similar, endogenous miRNAs.

The location of the targeted sequence on the template strand is defined by the location of the 5′ end of the targeted sequence. The 5′ end of the targeted sequence may be at any position of the transcription start site (TSS) core and the targeted sequence may start at any position selected from position 1 to position 5001 of the TSS core. For reference herein, when the 5′ most end of the targeted sequence from position 1 to position 2000 of the TSS core, the targeted sequence is considered upstream of the TSS and when the 5′ most end of the targeted sequence is from position 2002 to 5000, the targeted sequence is considered downstream of the TSS. When the 5′ most end of the targeted sequence is at nucleotide 2001, the targeted sequence is considered to be a TSS centric sequence and is neither upstream nor downstream of the TSS. For further reference, for example, when the 5′ end of the targeted sequence is at position 1600 of the TSS core, i.e., it is the 1600th nucleotide of the TSS core, the targeted sequence starts at position 1600 of the TSS core and is considered to be upstream of the TSS.

In some embodiments, the targeted sequence is located within a TSS core of the template stand. A “TSS core” or “TSS core sequence” as used herein, refers to a region between 2000 nucleotides upstream and 2000 nucleotides downstream of the TSS (transcription start site). Therefore, the TSS core comprises 4001 nucleotides and the TSS is located at position 2001 from the 5′ end of the TSS core sequence.

In some embodiments, the targeted sequence is located between 3000 nucleotides upstream and 3000 nucleotides downstream of the TSS.

In some embodiments, the targeted sequence is located between 2000 nucleotides upstream and 2000 nucleotides downstream of the TSS.

In some embodiments, the targeted sequence is located between 1000 nucleotides upstream and 1000 nucleotides downstream of the TSS.

In some embodiments, the targeted sequence is located between 500 nucleotides upstream and 500 nucleotides downstream of the TSS.

In some embodiments, the targeted sequence is located between 250 nucleotides upstream and 250 nucleotides downstream of the TSS.

In some embodiments, the targeted sequence is located between 100 nucleotides upstream and 100 nucleotides downstream of the TSS.

In some embodiments, the targeted sequence is located between 10 nucleotides upstream and 10 nucleotides downstream of the TSS.

In some embodiments, the targeted sequence is located between 5 nucleotides upstream and 5 nucleotides downstream of the TSS.

In some embodiments, the targeted sequence is located between 1 nucleotide upstream and 1 nucleotide downstream of the TSS.

In some embodiments, the targeted sequence is located upstream of the TSS in the TSS core. The targeted sequence may be less than 2000, less than 1000, less than 500, less than 250, less than 100, less than 10 or less than 5 nucleotides upstream of the TSS.

In some embodiments, the targeted sequence is located downstream of the TSS in the TSS core. The targeted sequence may be less than 2000, less than 1000, less than 500, less than 250, less than 100, less than 10 or less than 5 nucleotides downstream of the TSS.

In some embodiments, the targeted sequence is located +/−50 nucleotides surrounding the TSS of the TSS core. In some embodiments, the targeted sequence substantially overlaps the TSS of the TSS core. In some embodiments, the targeted sequence overlaps begin or end at the TSS of the TSS core. In some embodiments, the targeted sequence overlaps the TSS of the TSS core by 1, 2, 3, 4, 5, 6, 7, 8, 9, 10, 11, 12, 13, 14, 15, 16, 17, 18 or 19 nucleotides in either the upstream or downstream direction.

The location of the targeted sequence on the template strand is defined by the location of the 5′ end of the targeted sequence. The 5′ end of the targeted sequence may be at any position of the TSS core and the targeted sequence may start at any position selected from position 1 to position 4001 of the TSS core. For reference herein, when the 5′ most end of the targeted sequence from position 1 to position 2000 of the TSS core, the targeted sequence is considered upstream of the TSS and when the 5 ‘ most end of the targeted sequence is from position 2002 to 4001, the targeted sequence is considered downstream of the TSS. When the 5’ most end of the targeted sequence is at nucleotide 2001, the targeted sequence is considered to be a TSS centric sequence and is neither upstream nor downstream of the TSS. For further reference, for example, when the 5′ end of the targeted sequence is at position 1600 of the TSS core, i.e., it is the 1600th nucleotide of the TSS core, the targeted sequence starts at position 1600 of the TSS core and is considered to be upstream of the TSS.

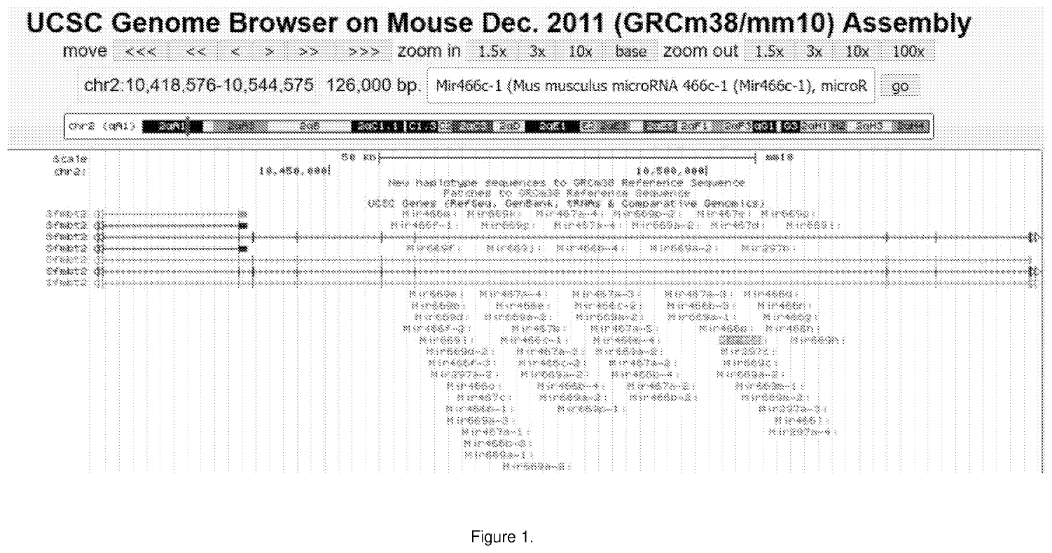

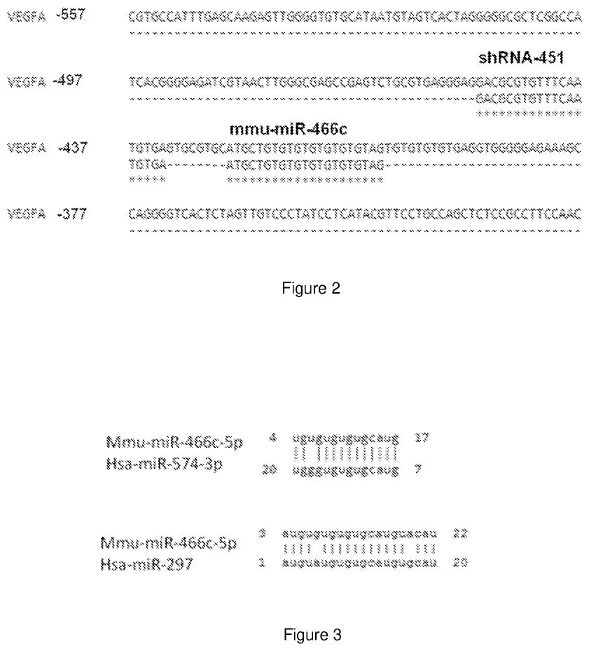

In accordance with the inventors' findings, it was hypothesized that the endogenous miRNAs may execute similar functions as the synthetic shRNAs in the regulation of Vegfa. In order to investigate it, next generation sequencing of miRNAs from nuclear and cytoplasmic fractions of hypoxic mouse endothelial cells was performed. The results showed that many miRNAs actually localize to the nucleus in response to hypoxia. Using then bioinformatic analyses, the present inventors could identify that a nuclear enriched mmu-miR-466c had putative binding sites within the murine Vegfa promoter (FIG. 2). mmu-miR-466c is encoded in a rodent-specific miRNA cluster in an intronic region, specifically in intron 10, of the Scm-like with four mbt domains 2 (Sfmbt2) gene (FIG. 1).

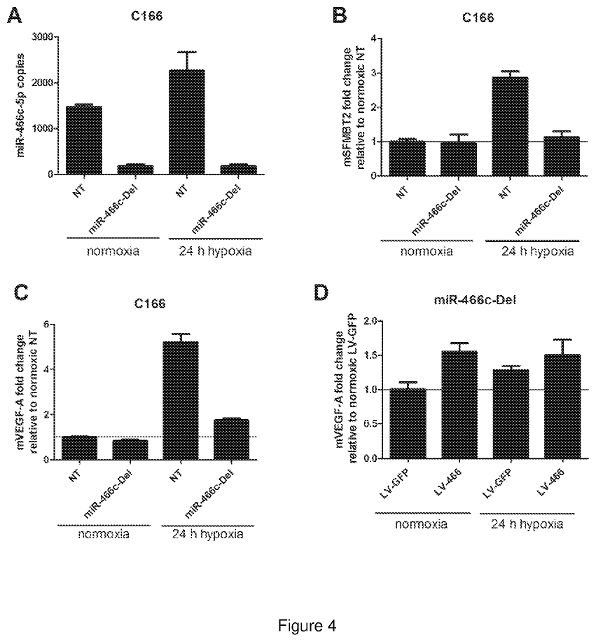

Sfmbt2 gene, like the miRNAs of interest, was observed to be responsive to hypoxia and significantly upregulated after 24 h of hypoxia treatment (FIG. 4). The obtained data suggests that mmu-miR-466c might collaborate in the fine-tuning of Vegfa expression during hypoxia by targeting its promoter.

In the bioinformatics analyses, the putative target site of mmu-miR466c was observed to be surprisingly close to the binding sites of the shRNA used previously for Vegfa gene activation (example in FIG. 2). For investigation of the possible mechanism of interaction between the miRNA and the promoter sequence, targeted-RNA sequencing was performed. The results identified an antisense RNA transcript on the mouse Vegfa promoter in C166 endothelial cells. This promoter-associated non-coding transcript is likely to be the target of the miRNAs and shRNAs in the Vegfa promoter.

The identification of miRNAs in a wide variety of organisms suggests an evolutionary conservation of miRNA regulation mechanism, and many specific miRNAs have been shown to be conserved between e.g. rodents and humans. Also, there might exist homologous miRNAs of mmu-miR-466c within the human genome. When scanning the human genome in the search of similar counterparts, the present inventors found that two microRNAs (hsa-miR-297 and hsa-miR-574-3p) shared an especially high amount of sequence similarity with mmu-miR-466c (FIG. 3). Due to the high similarity shared between the mouse miRNAs and human miRNAs, it is reasonable to assume that the human miRNA counterparts have similar targets and that mouse miRNAs transfected into human cells are able regulate human VEGFA, VEGFD and HIF1A mRNA levels.

The obtained data suggest that may be the microRNAs act at both transcriptional and post-transcriptional level targeting promoter associated non-coding transcripts as well as the 3′UTR region of the Vegfa. This would support the hypothesis that these miRNAs act in fine-tuning the tightly controlled Vegfa expression in hypoxic conditions.

In the present project, for further investigating the mechanism of action of mmu-miR-466c, the present inventors performed two different experiments: I) the present inventors transfected synthetic mmu-miR-466c mimics into human endothelial cell line Ea.hy926 and human epithelial cell line ARPE19 and observed the effects on the VEGFA mRNA levels. II) Next, for investigating the function of mmu-miR-466c, the present inventors established a stable mmu-miR-466c deletion mouse cell line using CRISPR/Cas9 genome editing tool.

The studied miRNA in this project (mmu-miR-446c) has been previously observed by the present inventors to tightly control murine Vegfa expression in response to hypoxia. Due to the high evolutionary conservation of miRNA regulatory mechanisms between organisms, and the existence of a human similar miRNA counterpart of mmu-miR-466c, the present inventors investigated how synthetic mmu-miR-466c mimics regulated VEGFA mRNA levels in human cell lines.

After transfecting the human endothelial cell line Ea.hy926 with a hairpin mimic of miR-466c (miR-466c 3p mim), (see examples for details) the present inventors found a significant downregulation in the VEGFA mRNA levels. Same downregulation effect was observed when transfecting the single stranded 3p mimic (ssRNA-466c-3p) and the duplex structure of this miRNA which included the 3p strand and 5p strand annealed. These results suggest that there might exist a strand bias selection in human endothelial cells and the 3p strand of the mmu-miR-466c might be functional downregulating VEGFA.

Moreover, after testing synthetic mimics of mmu-miR-466c in a human epithelial cell line, the present inventors could see a clear difference in the VEGFA regulation performance between the different types of mimics. The hairpin mimics (miR-466c-3p/5p mim) were able to strongly upregulate VEGFA, whereas the rest of mimics were inducing slightly changes or no changes in VEGFA mRNA levels. The big difference observed between mimics performances could be explained due to the hairpin mimics structure. The hairpin structure could be more alike the endogenous natural mmu-miR-466c and requires the processing in the cell and the activation of the miRNA processing complexes, whereas the other mimics single stranded or duplex form are already a more mature form, thus possibly not always entering the proper processing pathway.

The results using murine miR-466c mimics showed that miR-466c was able to regulate human VEGFA mRNA levels and, in addition, that human cells may be able to process the non-mature forms of the murine miRNA to generate a functional mature strand. Moreover, miR-466c seems to play opposite regulatory effects in the different epithelial and endothelial human cell lines tested. This is in accordance with the results obtained previously by the present inventors when testing shRNAs in different cell lines resulted in different effects on the Vegfa levels. The present inventors found previously that mmu-miR-466c is necessary for the induction of Vegfa expression under hypoxia in mouse endothelial cells, whereas in human endothelial cells it seems to have a down regulatory effect. Thus, the effects of miR-466c synthetic mimics in the VEGFA levels seem to be dependent on the cell type tested. The strong effect observed on VEGFA downregulation in the endothelial human cell line studied could be investigated in the future as an anti-angiogenic therapy.

Taken together, this data corroborates the potential of miRNAs for regulating gene expression in mammalian cells. The results are promising and increasing evidence points to miRNAs nuclear functions and gene regulatory roles. RNA-molecules have been proposed to be the earliest form of life on earth, preceding DNA and proteins. It is noteworthy to recognize that these miRNAs and small non-coding RNAs presented in this application contain large amount of repetitive dinucleotide sequences. The earliest forms of RNA could have had minimal amount of variation in their sequence, containing only two different nucleotides. Since DNA did not exist yet there was no need for variable DNA-coding sequences. Therefore, we propose that these small, repetitive sequence containing, RNAs as presented herein may be regulatory molecules from ancient biology.

An object of the invention is a method for treating a disorder caused by an insufficient amount of a VEGFA polypeptide by at least one cell in a subject comprising administering to said subject an effective amount of an miRNA composition comprising miRNA with at least 80% identity to at least one sequence according to SEQ ID NO:5, SEQ ID NO:6, SEQ ID NO: 3, SEQ ID NO:4, SEQ ID NO:15 or SEQ ID NO:16 to increase at least one activity of a VEGFA polypeptide on said cell.

A method for enhancing wound healing by increasing production of a VEGFA polypeptide by at least one cell in a subject comprising administering to said subject an effective amount of an miRNA composition comprising miRNA with at least 80% identity to at least one sequence according to SEQ ID NO:5, SEQ ID NO:6, SEQ ID NO: 3, SEQ ID NO:4, SEQ ID NO:15 or SEQ ID NO:16 to increase at least one activity of a VEGFA polypeptide on said cell, thereby increasing the occurrence of VEGFA-mediated prolonged or abortive wound healing is another aspect of this invention.

Still another aspect of the invention is a method of increasing angiogenesis in a tissue, comprising administering to said tissue an effective amount of an miRNA composition comprising miRNA with at least 80% identity to at least one sequence according to SEQ ID NO:5, SEQ ID NO:6, SEQ ID NO: 3, SEQ ID NO:4, SEQ ID NO:15 or SEQ ID NO:16 to increase at least one activity of a VEGFA polypeptide on said tissue.

“Angiogenesis” is defined herein as the growth or remodeling of vascular structures. Angiogenesis can be diagnosed or determined by in vivo and in vitro methods including MRI, angiograms, and histochemistry.

A preferred aspect of the invention is a method for treating a disorder caused by an underproduction of a VEGFA polypeptide, wherein said disorder relates to angiogenesis. In a more preferred aspect the angiogenesis is vasculogenesis. In an even more preferred aspect said disorder is a fibrotic disorder that is selected from the group consisting of injection fibrosis, endomyocardial fibrosis, mediastinal fibrosis, myleofibrosis, retroperiotoneal fibrosis, progressive massive fibrosis, nephrogenic systemic fibrosis, interstitial lung disease (ILD), idiopathic pulmonary fibrosis (IPF), scleroderma, radiation-induced pulmonary fibrosis, bleomycin lung, sarcoidosis, silicosis, pulmonary fibrosis, familial pulmonary fibrosis, nonspecific interstitial pneumonitis, autoimmune disease, renal graft transplant fibrosis, heart graft transplant fibrosis, liver graft transplant fibrosis, scarring, glomerulonephritis, cirrhosis of the liver, systemic sclerosis, or proliferative vitreoretinopathy.

A more preferred aspect of the invention is a method for treating a disorder caused by a too low amount of a VEGFA polypeptide, wherein said disorder is a cardiovascular disease. Said cardiovascular disease can be selected from the group consisting of heart disease, myocardial ischemia, heart failure and peripheral vascular disease.

“Heart disease” refers to acute and/or chronic cardiac dysfunctions. Heart disease is often associated with a decrease in cardiac contractile function and may be associated with an observable decrease in blood flow to the myocardium (e.g., as a result of coronary artery disease). Manifestations of heart disease include myocardial ischemia, which may result in angina, heart attack and/or congestive heart failure.

“Myocardial ischemia” is a condition in which the heart muscle does not receive adequate levels of oxygen and nutrients, which is typically due to inadequate blood supply to the myocardium (e.g., as a result of coronary artery disease).

“Heart failure” is clinically defined as a condition in which the heart does not provide adequate blood flow to the body to meet metabolic demands. Symptoms include breathlessness, fatigue, weakness, leg swelling, and exercise intolerance. On physical examination, patients with heart failure tend to have elevations in heart and respiratory rates, rales (an indication of fluid in the lungs), edema, jugular venous distension, and, in many cases, enlarged hearts. Patients with severe heart failure suffer a high mortality; typically 50% of the patients die within two years of developing the condition. In some cases, heart failure is associated with severe coronary artery disease (“CAD”), typically resulting in myocardial infarction and either progressive chronic heart failure or an acute low output state, as described herein and in the art. In other cases, heart failure is associated with dilated cardiomyopathy without associated severe coronary artery disease.

“Peripheral vascular disease” refers to acute or chronic dysfunction of the peripheral (i.e., non-cardiac) vasculature and/or the tissues supplied thereby. As with heart disease, peripheral vascular disease typically results from an inadequate blood flow to the tissues supplied by the vasculature, which lack of blood may result, for example, in ischemia or, in severe cases, in tissue cell death. Aspects of peripheral vascular disease include, without limitation, peripheral arterial occlusive disease (PAOD) and peripheral muscle ischemia. Frequently, symptoms of peripheral vascular disease are manifested in the extremities of the patient, especially the legs.

A further aspect of the invention is a method of decreasing haemorrhage in a tissue, comprising administering to said tissue an effective amount of an miRNA composition comprising miRNA with at least 80% identity to at least one sequence according to SEQ ID NO:5, SEQ ID NO:6, SEQ ID NO: 3, SEQ ID NO:4, SEQ ID NO:15 or SEQ ID NO:16 to increase the activity of a VEGFA polypeptide on said tissue.

A method of increasing endothelial cell proliferation in a tissue, comprising administering to said tissue an effective amount of an miRNA composition comprising miRNA with at least 80% identity to at least one sequence according to SEQ ID NO:5, SEQ ID NO:6, SEQ ID NO: 3, SEQ ID NO:4, SEQ ID NO:15 or SEQ ID NO:16 to increase the activity of a VEGF polypeptide on said tissue is an aspect of the invention.

Also a method of increasing wound healing in a tissue, comprising administering to said tissue an effective amount of an miRNA composition comprising miRNA with at least 80% identity to at least one sequence according to SEQ ID NO:5, SEQ ID NO:6, SEQ ID NO: 3, SEQ ID NO:4, SEQ ID NO:15 or SEQ ID NO:16 to increase the activity of a VEGF polypeptide on said tissue is an aspect of the invention.

An aspect of the invention is a synthetic oligonucleotide having at least 80% sequence identity to SEQ ID NO:5, SEQ ID NO:6, SEQ ID NO:3, SEQ ID NO: 4, SEQ ID NO: 15 or SEQ ID NO: 16 or a complementary sequence thereof. More specifically, the synthetic oligonucleotide is at least 85%, 90%, 95%, 96%, 97%, 98%, 99% or 100% identical to miRNA according to SEQ ID NO:5, SEQ ID NO:6, SEQ ID NO:3, SEQ ID NO: 4, SEQ ID NO: 15 or SEQ ID NO: 16.

A further aspect of the invention is a method for the diagnosis of a cardiovascular disease, wherein the method comprises the steps:

(a) providing a sample from an individual suspected of suffering from a cardiovascular disease;

(b) measuring the expression level of at least one sequence selected from SEQ ID NO: 15 and SEQ ID NO: 16 in the sample;

wherein an increased or reduced expression level of at least one sequence selected from SEQ ID NO: 15 and SEQ ID NO: 16 compared to a control sample indicates a heart disease or a prevalence or predisposition to a cardiovascular disease.

One preferred aspect of the invention is the synthetic nucleic acid molecule as disclosed above, which is single-stranded. Another preferred aspect of the invention is the synthetic nucleic acid molecule as disclosed above, which is at least partially double-stranded. One preferred aspect of the invention is the synthetic nucleic acid molecule as disclosed above, which is selected from RNA or DNA molecules. Another preferred aspect of the invention is the synthetic nucleic acid molecule as disclosed above comprising at least one modified nucleotide analog. In a still more preferred aspect the modification is selected from the group consisting of nucleobase modifications, sugar modifications, inter-sugar linkage modifications, backbone modifications, and any combinations thereof.

Still a further aspect of the present invention is a method of treating a myocardial infarction or chronic heart failure in a subject, the method comprising:
(a) identifying and selecting a subject in need of treatment for a myocardial infarction or chronic heart failure; and
(b) administering to the selected subject a therapeutically effective amount of a microRNA mmu-miR-466c or hsa-miR-297 or hsa-miR-574-3p, wherein miR-466c comprises at least 18, 19, or 20 nucleotides with at least 80% identity to SEQ ID NO: 5, SEQ ID NO:6, SEQ ID NO: 3 or SEQ ID NO: 4, including a region with 100% identity to nucleotides 1-8 of SEQ ID NO: 5, SEQ ID NO:6SEQ ID NO: 3 or SEQ ID NO: 4, wherein miR-297 comprises at least 18, 19, or 20 nucleotides with at least 80% identity to SEQ ID NO: 15, including a region with 100% identity to nucleotides 1-8 of SEQ ID NO: 15 and wherein miR-574-3p comprises at least 18, 19, or 20 nucleotides with at least 80% identity to SEQ ID NO: 16, including a region with 100% identity to nucleotides 1-8 of SEQ ID NO: 16.

A method of treating or preventing myocardial infarction, cardiac remodelling, or heart failure in a subject in need thereof comprising modulating the expression or activity of one or more miRNAs according to SEQ ID NOs: 3, 4, 5, 6, 15 or 16 or a homolog thereof in the heart cells of the subject is an aspect of the present invention.

A synthetic miRNA molecule having at least 80% sequence identity to SEQ ID NO: 5, SEQ ID NO: 6, SEQ ID NO: 3, SEQ ID NO: 4, SEQ ID NO: 15 or SEQ ID NO: 16 ora complementary sequence thereof is an aspect of the present invention.

According to one preferred aspect of the invention the synthetic miRNA molecule has at least 80% sequence identity to SEQ ID NO: 3.

According to one preferred aspect of the invention the synthetic miRNA molecule has at least 80% sequence identity to SEQ ID NO: 4.

According to one preferred aspect of the invention the synthetic miRNA molecule has at least 80% sequence identity to SEQ ID NO: 5.

According to one preferred aspect of the invention the synthetic miRNA molecule has at least 80% sequence identity to SEQ ID NO: 15.

According to one preferred aspect of the invention the synthetic miRNA molecule has at least 80% sequence identity to SEQ ID NO: 16.

According to one preferred aspect of the invention the synthetic miRNA molecule has at least 80% sequence identity to SEQ ID NO: 6.

A more preferred aspect of the invention is the synthetic miRNA molecule having at least 85% sequence identity to SEQ ID NO: 5, SEQ ID NO: 6, SEQ ID NO: 3, SEQ ID NO: 4, SEQ ID NO: 15 or SEQ ID NO: 16 or a complementary sequence thereof.

A more preferred aspect of the invention is the synthetic miRNA molecule having at least 90% sequence identity to SEQ ID NO: 5, SEQ ID NO: 6, SEQ ID NO: 3, SEQ ID NO: 4, SEQ ID NO: 15 or SEQ ID NO: 16 or a complementary sequence thereof.

A still more preferred aspect of the invention is the synthetic miRNA molecule having at least 95% sequence identity to SEQ ID NO: 5, SEQ ID NO: 6, SEQ ID NO: 3, SEQ ID NO: 4, SEQ ID NO: 15 or SEQ ID NO: 16 or a complementary sequence thereof.

A still more preferred aspect of the invention is the synthetic miRNA molecule having nucleic acid sequence comprising SEQ ID NO: 5, SEQ ID NO: 6, SEQ ID NO: 3, SEQ ID NO: 4, SEQ ID NO: 15 or SEQ ID NO: 16 or a complementary sequence thereof.

A further aspect of the present invention is the synthetic miRNA molecule as disclosed above, wherein said nucleic acid molecule is 14-30 nucleotides in length. The synthetic miRNA molecule as disclosed above, wherein the seed sequence comprises GAUGUGU or UACAUAC or UGUAUGUG, is also a further aspect of the invention. The most preferred seed sequence comprises GAUGUGU. Also, the synthetic miRNA molecule according to the invention, wherein said nucleic acid molecule binds to promoter region or 3′UTR of the target gene is a further aspect of the invention. One preferred embodiment of the invention is the synthetic miRNA molecule according to the invention, wherein the target gene is VEGFA, VEGFD or HIF1A. In the most preferred embodiment the target gene of the synthetic miRNA molecule is VEGFA.

According to a more preferred embodiment the synthetic miRNA molecule according to the invention comprises at least one modified nucleotide analog. According to a still one preferred embodiment the synthetic miRNA molecule is for use in the treatment of cardiovascular diseases.

An aspect of the present invention is a pharmaceutical composition comprising at least one synthetic miRNA molecule according to the invention and a carrier or vehicle. In the more preferred aspect, the carrier or vehicle of said pharmaceutical composition is suitable for therapeutic applications. According to a still more preferred aspect of the invention the pharmaceutical composition further comprises at least one pharmaceutically acceptable excipient. According to the most preferred embodiment the pharmaceutical composition is for use in the treatment of cardiovascular diseases.

A recombinant expression vector comprising at least one synthetic miRNA molecule of the present invention is an aspect of the invention. A cell comprising this recombinant expression vector is another aspect of the invention.

A method of modulating the expression of a target protein in human, comprising administering the synthetic miRNA molecule of the invention to a subject in need thereof is an aspect of the invention. A more preferred aspect of the invention is this method, wherein the expression of the target protein is increased. According to a still more preferred aspect of the invention the expression of the target protein is increased by at least 30%. According to the most preferred embodiment the target protein is increased by at least 50%.

According to another preferred embodiment, the method comprises administering to the subject an effective amount of at least one synthetic miRNA having at least 80% sequence identity to SEQ ID NO: 5, SEQ ID NO: 6, SEQ ID NO: 3, SEQ ID NO:4, SEQ ID NO: 15 or SEQ ID NO: 16. In a still more preferred embodiment the miRNAs to be administered have at least 90% sequence identity to said sequences. In a still more preferred embodiment the miRNAs to be administered have at least 95% sequence identity to said sequences. In a still more preferred embodiment the miRNA to be administered comprises sequence according to SEQ ID NO: 5, SEQ ID NO: 6, SEQ ID NO: 3, SEQ ID NO:4, SEQ ID NO: 15 or SEQ ID NO: 16.

An aspect of the present invention is a method for determining whether a subject is at risk of or suffering from a cardiovascular disease, the method comprising the steps of:

(A) providing a sample of an individual who is suspected of cardiovascular disease;

(B) optionally extracting RNA from said sample;

(C) measuring the expression level of hsa-miR-297 or hsa-miR-574-3p by qRT-PCR; wherein a decreased level of expression of hsa-miR-297 or hsa-miR-574-3p compared to a control sample indicates a presence of or a predisposition to cardiovascular disease.

### EXAMPLES

Cell Culture

Murine C166 (yolk-sac derived mouse endothelial cell line, ATCC: CRL-2581) cells, human EA.hy926 (somatic cell hybrid endothelial cells, ATCC: CRL-2922) and human ARPE-19 (retinal epithelial cells, ATCC: CRL-2302) cells were maintained under normal conditions (37° C., 5% CO₂) in Dulbecco's Modified Eagle's Medium (DMEM) (Sigma-Aldrich) containing 10% fetal bovine serum (FBS) and 1% penicillin-streptomycin (P/S).

C166 CRISPR/Cas9 transfected cells were maintained right after cell sorting under normal conditions (37° C., 5% CO₂) in DMEM containing 20% FBS and 1% P/S.

During the hypoxia experiments performed using C166 cell line, cells were cultured in normal media but under hypoxic conditions (1% 02, 5% CO₂).

Constructing Lentiviral Mmu-miR-466c Expression Vector

Third generation human immunodeficiency virus 1 (HIV-1)-based LV-PGK-GFP-U6-RNA vectors were used for overexpression of mmu-miR-466c. mmu-miR-466c was cloned into the vector by PCR cloning using cloning primers (SEQ ID NOs: 7 and 8) as a hairpin in the genomic context (total of 255 bp fragment). As a control, we used lentivirus (LV) encoding only GFP. The vectors were prepared by standard calcium phosphate transfection method in 293T cells.

Constructing miRNA Mimics

miRNA mimics were generated for the endogenous miRNAs mmu-miR-466c-5p and mmu-miR-466c-3p, as well as for synthetic versions of miR-466c-5p (fusion mimic and repeat mimic) (SEQ ID NOs: 3-6). The desired miRNA sequences were provided for Dharmacon, where the mimics were prepared according to their protocols for miRIDIAN microRNA mimics as disclosed in EP2261334A2.

Lentiviral Transductions

Third generation human immunodeficiency virus 1 (HIV-1)-based LV-PGK-GFP-U6-RNA vectors were used for overexpression of mmu-miR-466c. As a control, we used lentivirus (LV) encoding only GFP. The vectors were prepared by standard calcium phosphate transfection method in 293T cells. C166 cells were transduced with lentiviral vector expressing mmu-miR-466c (LV-466). Cells were transduced using MOI 10 and samples were collected 5 days after the transduction.

miRNA-466c Synthetic Mimics Transfections

In order to find the optimal transfection efficiency and lower rate of cell death for transfecting the miRNA mimics into a human cell line, the present inventors tested different ratios of transfection reagent/fluorescent siRNA into human retinal epithelial ARPE-19 cells. The best efficiency (99,83%) was achieved using 7.5 μL of Mirus TransIT-TKO® reagent and 30 pmol of the fluorescent siRNA (cat #AM4620, Ambion) and that was used later for transfecting also human EA.hy926 cells with the mimics.

For miRNA mimics transfections, EA.hy926 cells and ARPE-19 cells were seeded at a density of 7.5×10⁴ cells/well and 1.5×10⁵ respectively onto a 6-well plate. After 24 hours, cells were transfected with a total amount of 30 pmol/well of synthetic 466c-microRNA mimics or negative control siRNA (cat #AM4611, Ambion). For one well, miRNA mimics were combined with 7,5p1 of Mirus TransIT-TKO® reagent diluted in 250 μl of serum-free DMEM and incubated at room temperature for 15-30 minutes. Three different types of mimics were tested: i) hairpin structure mimic designed and chemically modified so that only 3p or 5p strand will be produced after the cellular processing (miR-466c-3p/5p mim; Dharmacon); ii) single stranded mimics of 3p and 5p (ssRNA-466c-3p/5p mim; IDT); iii) duplex structure mimic consisting of ssRNA-466c 3p and 5p strands annealed (duplex).

For the negative control transfection, Negative Control siRNA (cat #AM4611, Ambion) was used. Next, 48 hours after transfections, total RNA extraction was performed using TRI reagent (Molecular Research Center) according to manufacturer's instructions.

RNA Extraction, cDNA Synthesis and Quantitative RT-PCR

For gene expression analysis, total RNA was extracted from mimics treated cells and TSBs treated cells with TRI reagent (Molecular Research Center) according to the manufacturer's instructions and treated with DNAse I, RNAse-free kit (cat. #EN0523, Thermofisher Scientific). The obtained mRNA was reverse transcribed using RevertAid RT Reverse Transcription Kit (cat. #K1691, ThermoFisher Scientific) according to manufacturer's protocol. For expression analysis in EA.hy926 and ARPE19 samples, VEGFA and ACTB quantification was performed using TaqMan Gene Expression assays (mmu-miR-466c-3p ID: 464896_mat; mmu-miR-466c-5p ID: 463771_mat; VEGFA ID: Hs00173626_m1; ACTB ID: Hs01060665_g1; Life Technologies). For expression analysis in C166 samples, Vegfa and Actb quantification was performed using TaqMan Gene Expression assays (Vegfa ID: Mm00437306_m1; Actb ID: Mm_00607939_s1; Life Technologies). Standard curve was produced of single-stranded synthetic RNA molecules corresponding to the mature miRNA sequence in order to determine absolute miRNA copy numbers. For expression analysis of Vegfa, Sfmbt2, Yy1, and Hif1α, cDNA was synthesized using Transcriptor First Strand cDNA Synthesis Kit (Roche) and quantification performed using TaqMan Gene Expression Assays (Vegfa ID: Mm00437306_m1; Sfmbt2 ID: Mm00616783_m1; Yy1 ID: Mm00456392_m1; Hif1α ID: Mm01198376_m1; endogenous control Actb ID: Mm00607939_s1; Life Technologies).

Data Analysis

All analyses were performed using Prism 5 (GraphPad Software). Data were compared using an unpaired two-tailed Student's t-test. The significance level was set at P<0.05. Data are indicated as means±SD. The number of biologically independent experiments and P values are indicated either in the main text or in the figure legends.

### Example 1. Removal of Mmu-miR-466c Abolishes Vegfa Expression in Hypoxic Endothelial Cells

To analyze the role of mmu-miR-466 in the regulation of Vegfa in hypoxic response, CRISPR-mediated gene editing was used to remove 97 bp region from the Sfmbt2 intron 10 which contains the mmu-miR-466 hairpin. Vegfa expression is known to be upregulated upon hypoxia. After removal of mmu-miR-466, the normally observed induction of mmu-miR-466 expression in hypoxia was not present (FIG. 4A). The expression of the parent gene, Sfmbt2, was also not anymore induced in response to hypoxic stimuli (FIG. 4B). Importantly, the upregulation of Vegfa expression in response to hypoxia was also abrogated when miR-466 was removed (FIG. 4C). When miR-466 levels were restored using lentiviral transduction, levels of Vegfa rose equally both in normoxic and hypoxic conditions, but hypoxia did not further induce Vegfa expression (FIG. 4D).

### Example 2. Generation of Mmu-miR-466c Deletion Cell Line by CRISPR/Cas9

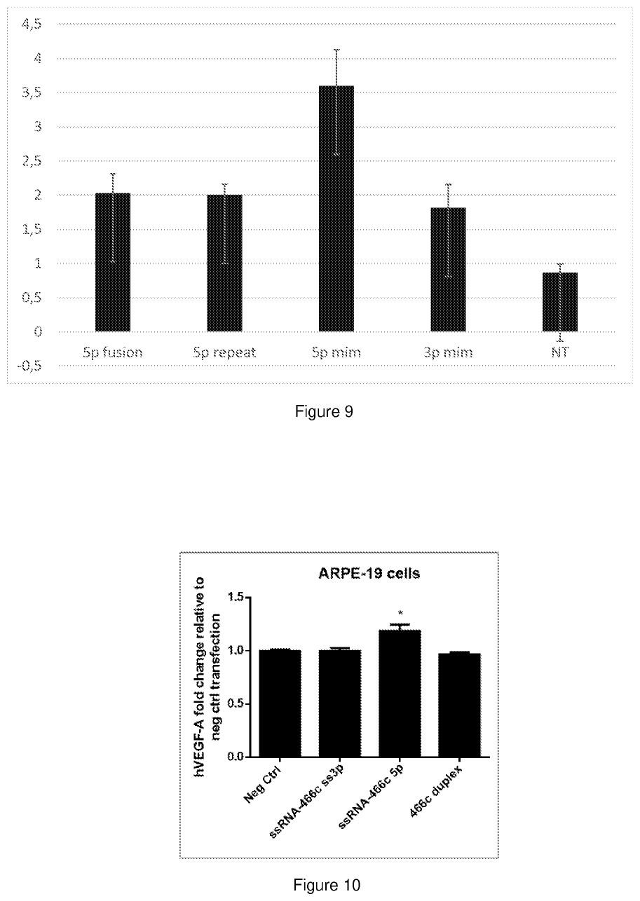

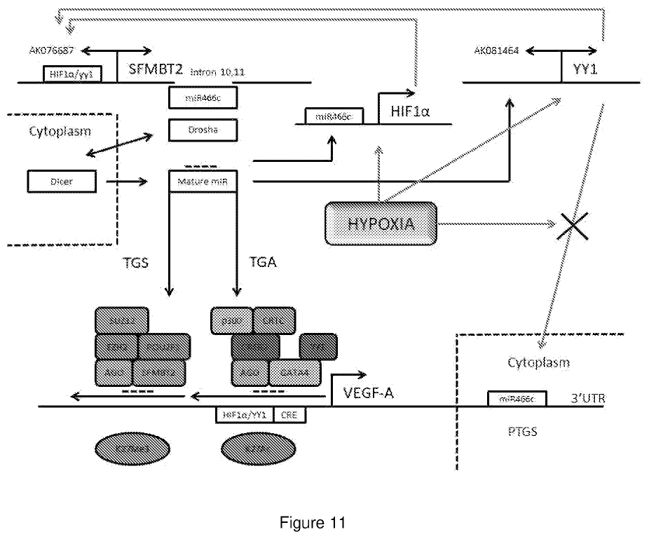

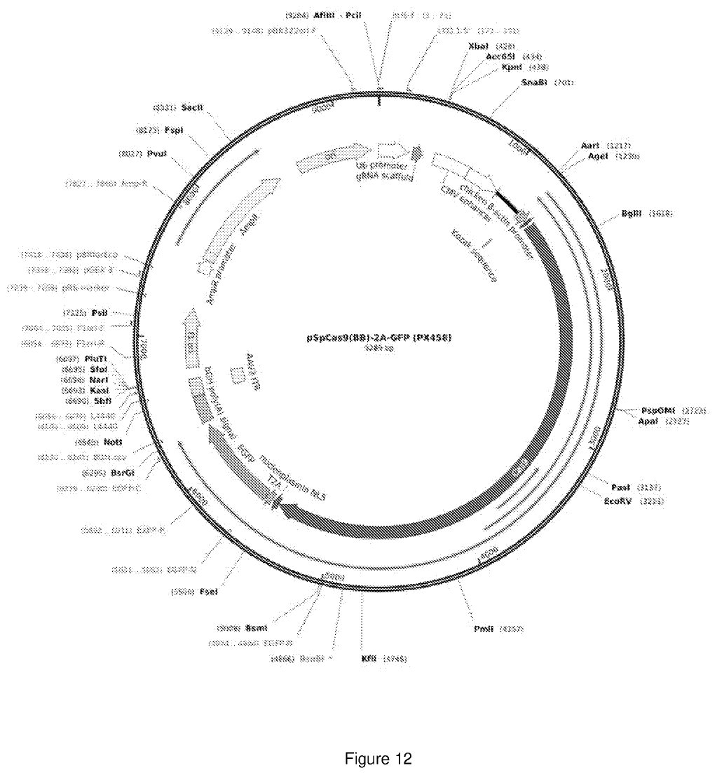

In order to remove mmu-miR-466c from intron 10 of Sfmbt2, two guide RNAs were cloned into separate expression plasmids (pcDNA-H1-sgRNA, see FIG. 9) and transfected into C166 cells along with Cas9 plasmid co-expressing GFP (PX458, cat. #48138, Addgene) using Nucleofector I (Amaxa). CRISPR was directed to the deletion side by guide oligos (SEQ ID NOs: 9-12) as disclosed for example in U.S. Pat. No. 8,697,359B1. Based on GFP positivity, single cells were sorted into 96-well plate wells using sorting FACS (BD FACSARIA III Cell Sorter) and clonal cell populations established. Cultures were genotyped by PCR to identify cells that contained the desired deletion, and positive clones further confirmed by Sanger sequencing.

TABLE 1
Forward and reverse guides designed to
target flanking regions of miR-466c.

| | Guide name | Sequence |
|---|---|---|
| miR-466c | pcDNA-H1- | 5′- AGAGTCAGGAAGATCAGGA-3′ |
| forward | sgRNA-466- | (SEQ ID NO: 7) |
| guide | Rev-1 | |
| miR-466c | pcDNA-H1- | 5′- CTAGCAAGCATTTTCACTC-3′ |
| reverse | sgRNA-466- | (SEQ ID NO: 8) |
| guide | Fwd-1 | |

TABLE 2
Primers used for genotyping miR-466c deletion.

| Primer name | Sequence |
|---|---|
| 466-del-fwd | 5′- TCAGGAGTGCAAGTTCATGGT -3′ |
| (forward primer) | (SEQ ID NO: 13) |
| 466-del-rev | 5′- GGATTGATGAGTGCCATTCCC -3′ |
| (reverse primer) | (SEQ ID NO: 14) |

### Example 3. Mmu-miR-466c Regulates VEGFA in Human Cell Lines

In order to test if murine miR-466c could have any effect in the mRNA levels of human VEGFA, the present inventors transfected human Ea.hy926 and ARPE-19 cells with synthetic miR-466c mimics.

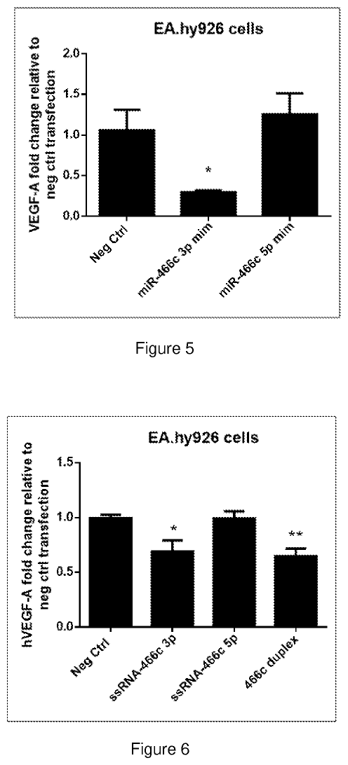

The t-student test performed in Ea.hy926 samples treated with the hairpin form miR-466c-3p mim revealed a significant decrease in VEGFA mRNA levels when compared to negative control transfection (negative control siRNA cat #AM4611, Ambion). (FIG. 5). miR-466c-3p mimic was able to downregulate VEGFA by inducing approximately a 3-fold downregulation (0.296 fold-change). Same effect was observed when treating the cells with single-stranded ssRNA-466c-3p mimic, which was able to downregulate VEGFA by inducing approximately a 1.42-fold downregulation (0.697 fold-change) when compared to negative control transfection (FIG. 6).

In contrast, when the hairpin mimic 5p (miR-466c-5p mim) and the single-stranded form mimic 5p (ssRNA-466c-5p) were tested, none showed any effect in VEGFA mRNA levels when compared to negative control samples (FIG. 5 and FIG. 6).

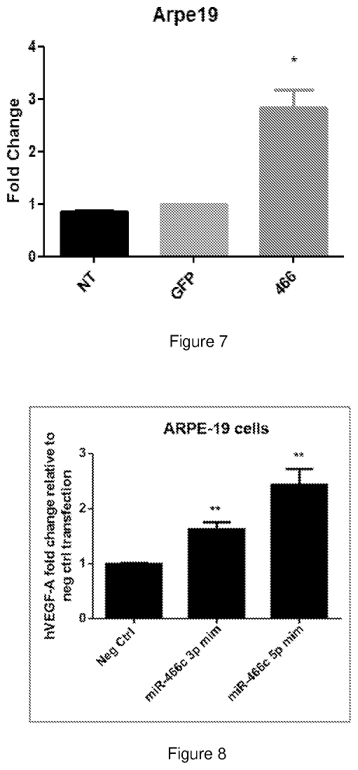

The present inventors next tested if the synthetic duplex form of mmu-miR-466c (466c duplex), consisting of single-stranded ssRNA-466c 3p and ssRNA-466c 5p mimics annealed, could exert similar functions as observed when the strands are transfected separately. When the synthetic duplex form was transfected into Ea.hy926 cells, VEGFA expression was significantly downregulated by 1.56-fold (0.64 fold-change) when compared to negative control transfection (FIG. 6). When ARPE-19 cells were transduced with lentiviral vector expressing mmu-miR-466 VEGFA expression was increased at 5 day timepoint as compared to controls (FIG. 7). The t-student test performed in ARPE-19 samples treated with the hairpin form mimic miR-466c-3p and miR-466c-5p revealed a significant upregulation of VEGFA mRNA levels when compared to negative control transfection (FIG. 8). Synthetic 5p-fusion and 5p-repeat RNAs increased VEGFA expression 2-fold in ARPE-19 cells as compared to NT control. miR-466c-5p mimic (5p mim) increased VEGFA expression 3,6-fold. (FIG. 9). miR-466c-3p mimic and miR-466c-5p were able induce a 1.62 and 2.43-fold change respectively in the mRNA levels of VEGFA, when compared to negative control.

Regarding the single stranded mimics, only the ssRNA-466c-5p was able to induce a slight upregulation in VEGFA expression of 1.18-fold-change compared to negative control. The ssRNA-466c-3p and the duplex structure did not induce any significant change in the expression of VEGFA (FIG. 10).

### Example 4. Cell Viability after miRNA Administration

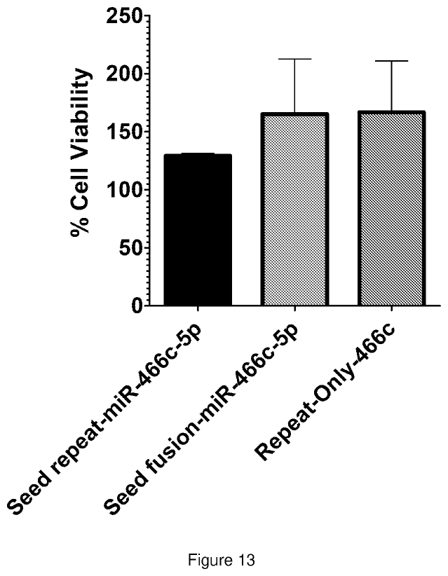

Cell viability after miRNA mimic transfection was assessed to measure the potential toxicity of miRNA for the cells. Cell viability was tested using MTT assay to see if the miRNA mimics induce changes in viability or proliferation of the cells. The sequences used in this experiment were according to SEQ ID:NOs 17-19, i.e. repeat only miR-466c, seed repeat miR-466c-5p and seed-fusion miR-466c-5p. None of the mimics reduced cell viability in ARPE-19 cell culture so no toxicity was observed after miRNA administration. In contrast, Fusion-miR-466c-5p-mimic and Repeat-miR-466c-5p increased cell viability compared to the control (commercial negative control siRNA). These results (FIG. 13) show that miR-466c-5p increases cell proliferation and shows regenerative potential.

ARPE-19 cells were transfected with miRNA mimics as described in “miRNA-466c synthetic mimics transfections” in Examples. Cell viability was measured 24 h after transfection using colorimetric MTT assay (Biotium, cat #30006) according to the manufacturer's protocol. MTT (3-(4,5-dimethylthiazol-2-yl)-2,5-diphenyltetrazolium bromide) assay is an established protocol to determine the amount of viable cells and differences in proliferation after drug administration measurement (Kumar et al, Cold Spring Harb Protoc. 2018 Jun. 1; 2018(6). doi: 10.1101/pdb.prot095505). Results are calculated in comparison to cells transfected with commercial negative control siRNA (Silencer™ Negative Control No. 1 siRNA, Thermo Fisher Scientific, cat #AM4611).

### Example 5. miRNA Packaging in Extracellular Vesicles

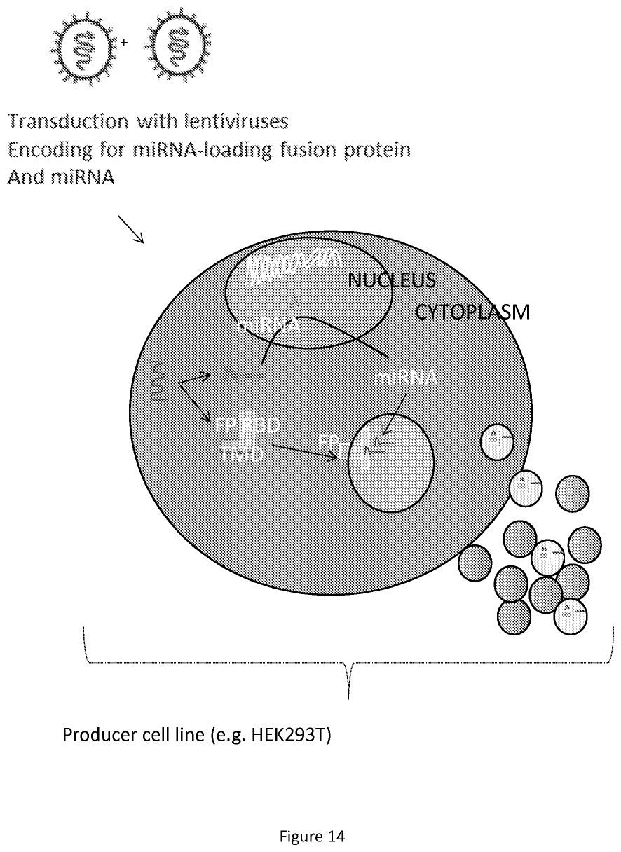

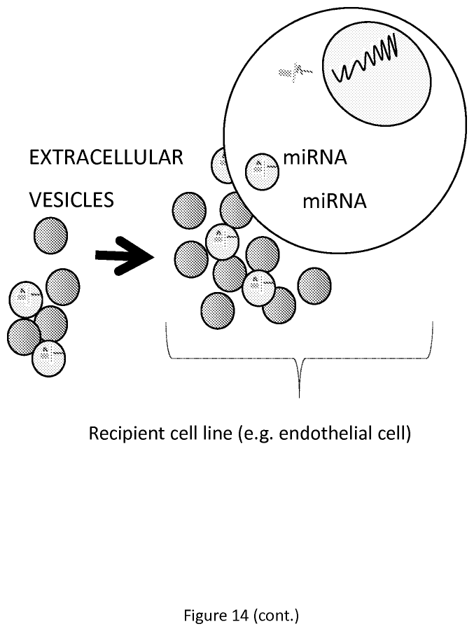

A schematic figure of miRNA packaging in extracellular vesicles is shown in FIG. 14. miRNA is more efficiently circulated in blood and taken to target tissue when it is packed inside delivery vehicle, such as extracellular vesicles (EVs). Delivery vehicles also protect RNA from degradation by endoribonucleases that are present in blood. EVs can be modified by overexpressing miRNA and modified EV-fusion proteins (FP) in producer cell line. Fusion protein consists of RNA-binding domain (RBD), from e.g. Ago2, and EV-specific transmembrane protein domain (TMD), from e.g. CD9 or TSG101 protein, that are fused together so that miRNA binds to the RNA-binding domain, which is connected to EV-specific transmembrane protein domain that directs the miRNA-fusion protein-complex to the EVs generated in the cell. In order to package therapeutic miRNA into EVs, both fusion protein and miRNA are overexpressed in a producer cell line. This increases the miRNA amount loaded inside each EV particle yielding a more concentrated therapy particle.

To obtain large quantities of miRNA-EV-drug, the producer cell line modified with overexpression of miRNA and EV-fusion protein is grown in iCELLis Nano bioreactor (Pall Corporation). (Fusion protein production was performed similarly to Valkama et al. Gene Ther. 2018 January; 25(1):39-46. doi: 10.1038/gt.2017.91. Epub 2017 Oct. 5.) This is more efficient than production on cell culture plates since one bioreactor has growth are of 4 m2 and volume 1 L. Cell culture medium containing the miRNA-EVs is collected and purified using TFF (concentration of medium, diafiltration to PBS) and chromatography (size exclusion columns to obtain more purified EV population), similarly as described in Nordin et al. (Methods Mol Biol. 2019; 1953:287-299. doi: 10.1007/978-1-4939-9145-7_18).

### Example 6. Fusion Protein Expression

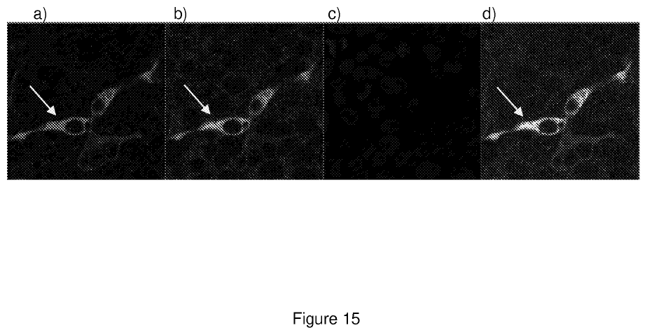

To package miRNAs efficiently into carrier EVs, fusion protein constructs were generated. Fusion protein included RNA-binding domain (Ago2) and EV-specific transmembrane protein domain (CD9 or TSG101) fused together to enhance the miRNA loading inside EVs. Immunofluorescence staining for RBD and TMD from transfected cells was performed to analyze the co-localization in the cells. Co-localization of these domains suggests that the two domains are fused together and not expressed separately in the cells. In fluorescent microscopy pictures we can see that both Ago2 and CD9 stain in the same regions (merge, on the right, FIG. 15d) in the cell cytoplasm (outside DAPI-stained cell nuclei). This indicates that the fusion protein is produced correctly.

Cloning of Fusion Proteins:

Fusion recombinant proteins (Human LV.CD9.hAgo2 and hTSG101.hAgo2) designed by the SnapGene software (TM1.1.3) and were constructed into the pLenti-hPGK backbone. The primers for cloning of the fusion proteins were according to SEQ ID NOs 20-31, respectively. humanAgo2 fragment was amplified from mCLOVER-NLS-AGO2 plasmid, gifted by Dr. Markus Hafner (Laboratory of Muscle Stem Cells and Gene Regulation). The CD9 fragment was amplified from HEK-293 cDNA and TSG101 was amplified from ARPE cell line's cDNA by High-Capacity cDNA Reverse Transcription Kit (Applied Biosystems™,4368814). The CloneAmp HiFi PCR Premix (Takara Bio USA, 639298) was used for PCR reaction and In-Fusion® HD Cloning Kit (Takara Bio USA, 638909) used for fusion and cloning of fragments according the manufacturers protocols.

Immunofluorescence:

Double immunofluorescence staining method was used for tracking of fused proteins, simultaneously. HEK-293 cells were cultured on the coverslip in the 6-well plate and constructed vectors transiently transfected via TransIT® Transfection Reagent Mirus. 48 hours after transfection, at 70-80% confluency, cells were rinsed by PBS to remove the dead cells and debris and fixed by paraformaldehyde (4% in PBS) for 15 min at room temperature (RT). Cells were washed three times with PBS. Permeabilization was done with 0.25% Triton X-100 for 15 min at RT and cells were rinsed three times for 5 min with PBS-0.05% Tween-20 (PBST). Unspecific binding sites were blocked with 10% normal goat serum (NGS) (S-26-LITER, Merck Millipore) in PBST for 30 minutes in gentle shaking in RT. The primary and secondary antibodies were diluted in staining buffer (5% NGS in PBST). For simultaneously double staining of fused proteins, two primary (e.g. primary anti-CD9 plus primary anti-Ago2) and two secondary antibodies (Alexa Fluor-555 plus Alexa Fluor-488) were diluted in the same tube and mixed. The cells were incubated with primary antibodies for 2 h in RT by gently inverting then washed three times with PBS for 5 minutes in RT. For secondary staining, the Alexa Fluor-555 (anti-rabbit, 4413S, Cell signalling) and Alexa Fluor-488 (Goat anti-mouse, 4408S, Cell signalling) were used. The cells were incubated with secondary antibodies for 1.5 hours by gentle shaking in RT and after three times washing with PBS, the coverslips were transferred to the microscope slide with 50 μl of mounting medium with DAPI (Vector Lab, H-1200) and were sealed with nail polish. Confocal laser scanning microscopy was performed using LSM700 Laser Scanning Confocal Microscope (LSM 700; Carl Zeiss Microscopy GmbH). Microscope configuration was the following: The objective lens; Plan-Apochromat 63X/1.40 oil M27. The sequential scanning with stack mode (0.5 μm for Z-scaling and 20.50 μm for stack size). The excitation: 405 nm (Blue: DAPI), 488 nm (Green: Alexa Fluor-488) and 555 nm (Red: Alexa Fluor-555). Results are shown in FIG. 15.

### Example 7. RNA Loading Capacity of Fusion Proteins

The efficiency of fusion proteins to load the miRNA inside EVs was assessed by isolating RNA from EVs collected from producer cell line transfected with fusion protein and miR-466c expression plasmids and quantifying miR-466c-5p levels by qPCR. Control group in the experiment was transfected with GFP plasmid instead of fusion protein plasmid, therefore determining the base level of miR-466c in EVs. RNA was isolated from the same number of EV particles for each treatment group as assessed by nanoparticle tracking analysis (NanoSight NS300). qPCR with Taqman microRNA assay for miR-466c-5p showed that fusion protein consisting of Ago2 and CD9 was more efficient that fusion protein consisting of Ago2 and TSG101 (qPCR Ct 22.4 and 23.8, respectively, where lower Ct indicates earlier amplification of miRNA, i.e. indicates that the sample contains more miRNA than sample with higher Ct-value). Both fusion protein transfections were shown to increase miR-466c-5p level in EVs compared to the control group (Ct 33.3), indicating that fusion protein expression significantly increases the miRNA loading to EVs. miR-466c-5p levels from fusion protein of Ago2 and CD9 transfection was detected 11 Ct's before control group, indicating 2000-fold increased miRNA packaging in EVs (theoretic calculation when the amount of DNA is duplicated in every cycle of PCR). Therefore, fusion protein assisted loading of miRNA results in more concentrated therapeutic levels (i.e. same EV particle number contains more miRNA, which is the active therapeutic agent).

Transfection of Fusion Protein and miRNA Plasmids and EV Isolation:

For isolation of EVs from conditioned media of HEK293 cell, the cells were cultured in complete media with 2% FBS. For EVs isolation, the cells were cultured (seeding density 4-4.5×106) in 15 cm plate. 15 μg fusion protein plasmid plus 15 μg shRNA expression plasmid, co-transfected via 45 μg PEI reagent (Alfa Aesar, cat #43896). PEI transfection protocol was adapted and modified from Longo et al (Methods Enzymol. 2013; 529:227-40. doi: 10.1016/6978-0-12-418687-3.00018-5). 15 μg LV-GFP expression plasmid plus 15 μg shRNA expression plasmid, co-transfected by 45 μg PEI as a negative control for each miRNA group. 4 hours after PEI transfection the media was changed with a fresh complete media. 48 hours after transfection, the condition media (17 ml per plate) was collected and centrifuged two times with 300 g and 2000 g for getting rid of dead cells and debris. The supernatant was filtered by 0.2 μm filter and filtered conditioned media was concentrated to 500 μl with Amicon® Ultra-15 centrifugal filter device (10 KDa cut-off). EVs were purified using qEV Column (Izon Science) according the manufacturers protocol. Fractions 7 to 10 were pooled (2 ml) and saved in −20° C. for further experiments. The engineered-particles were characterized by nano particle tracking analysis machine (NanoSight NS300).

RNA Isolation from EVs:

The total RNA from purified exosome was isolated by TRIzol (TRIzol® reagent; Invitrogen) and isopropanol precipitation method. Briefly, 750 pTRIzol reagent was added to maximum 200 μl EVs sample. To increase the efficiency of RNA precipitation, 1 μl GlycoBlue (AM9515, Invitrogen™) was added to the lysis sample and incubated at 80° C. for 5 min. After phenol step and three phase separation, the aqueous phase was mixed with 500 μl isopropanol and incubated at −20° C., overnight for RNA precipitation. The RNA pellet was diluted with 5p1 RNase free water and 5p1 precipitated RNA was used for miRNA specific cDNA synthesis. TaqMan-probe small RNA assay kit was used for reverse transcriptase reaction and qPCR of candidate miRNA. Specific single strand cDNA for candidate miRNA was synthesized according to the manufacturers protocol of the kit (Thermo Fisher Scientific Inc). The cycle threshold (Ct) values of reactions were plotted against fold change. The Ct values were for control 33.30551, for FP1 (Ago2/CD9) 22.48958 and for FP2 (Ago2/TSG101) 28.36981.

### Example 8. EV-Loaded miRNA in Recipient Cells

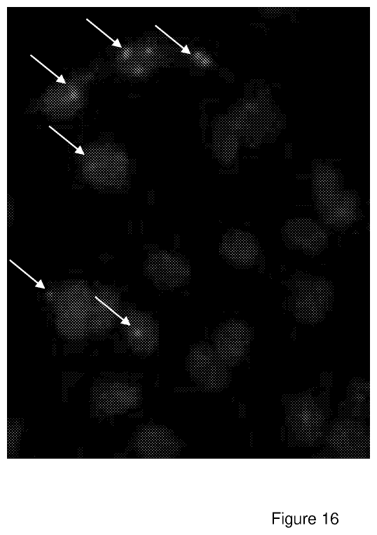

To observe if miRNA packaged in the EVs with fusion protein is taken up in recipient cell, miRNA levels in recipient cells were visualized using fluorescent in situ hybridization (FISH) (FIG. 16). EVs loaded with mmu-miR-466 were administered to human recipient cell line, which reduces the background since miR-466c is not found in human (rodent-specific). In the confocal microscopy images miR-466c-5p is stained and seen as dots, indicated by arrows in the figure. Importantly, miR-466c-5p is found in recipient cell nucleus. miR-466c-5p action we describe here is based on the nuclear role of miR-466c-5p targeting nuclear non-coding transcripts, hence the localization in the nucleus after administration to the cells indicates that the miRNA is taken up in the cells and transported from the cytoplasm to the nucleus to bind to its target transcripts.

miRNA FISH:

EVs isolated from producer cells expression fusion protein with Ago2 and TSG101 fusion and miR-466c were administered to ARPE-19 cells. Thermo Fisher Scientific ViewRNA™ miRNA ISH Cell Assay Kit (cat #QVCM0001) was used for miRNA FISH together with Thermo Fisher Scientific ViewRNA Cell Plus Probe Set: catalog #VM-06 (S-koko), assay ID: VM1-28918-VCP according to manufacturer's protocol.

1. A synthetic miRNA molecule having at least 80% sequence identity to SEQ ID NO: 5 or SEQ ID NO: 6, wherein said synthetic miRNA molecule comprises a seed sequence GAUGUGU.

2. The synthetic miRNA molecule of claim 1, wherein said nucleic acid molecule is 14-30 nucleotides in length.

3. The synthetic miRNA molecule of claim 1, wherein said nucleic acid molecule binds to promoter region or 3′UTR of the target gene.

4. The synthetic miRNA molecule of claim 3, wherein the target gene is VEGFA, VEGFD or HIF1A.

5. The synthetic miRNA molecule of claim 4, wherein the target gene is VEGFA.

6. The synthetic miRNA molecule of claim 1 for use in the treatment of cardiovascular diseases.

7. A pharmaceutical composition comprising at least one synthetic miRNA molecule of claim 1 and a carrier or vehicle.

8. The pharmaceutical composition of claim 7, wherein the carrier or vehicle is a pharmaceutically acceptable carrier or vehicle is suitable for therapeutic applications.

9. The pharmaceutical composition of claim 7 for use in the treatment of cardiovascular diseases.

10. A recombinant expression vector comprising at least one synthetic miRNA molecule of claim 1.

11. A cell comprising the recombinant expression vector of claim 10.

12. A method of modulating the expression of a target protein in human, comprising administering the synthetic miRNA molecule of claim 1.

13. The method of claim 12, wherein the expression of the target protein is increased.

14. The method of claim 12, wherein the method comprises administering to the subject an effective amount of at least one synthetic miRNA having at least 80% sequence identity to SEQ ID NO: 5 or SEQ ID NO: 6.

15. The synthetic miRNA molecule of claim 1 for use in modulating the expression of a target protein in human.

16. The synthetic miRNA molecule for use according to claim 15, wherein the expression of the target protein is increased.

17. The synthetic miRNA molecule for use according to claim 15, wherein an effective amount of at least one synthetic miRNA having at least 80% sequence identity to SEQ ID NO: 5 or SEQ ID NO: 6 is administered to a subject.

18. A method of treating a cardiovascular disease in a human in need of treatment thereof, the method comprising administering the synthetic miRNA molecule of claim 1 to the human.

19. The method of claim 18, wherein the method comprises administering to the subject an effective amount of at least one synthetic miRNA having at least 80% sequence identity to SEQ ID NO: 5 or SEQ ID NO: 6.


SEQUENCE LISTING (33 sequences)

SEQ ID NO: 1 - DNA, 5001 bp, Mus musculus
  mouse VEGF promoter nucleotide sequence
tcctctacctttcaaattctggggttacaagcatgtggtcccttcccatccccttcccttctttcacctaggtgtggagtatatctcttctcccagccctccctgactctagccagatcatattggcttgggaaatgaaccagataagcacacgtacatcaactcttacacctctctacaaccctggcaggaggcaacaacactttccatttcacagatgagaagactgaggctcacagctccggaaatagtgaaccagatttgtgctccgtctgccatgttttcccattcctcggtatgggttgggtgggggtgtggggtgtagcagaagccaaggctcttcacagtgagggatcaccaaccagtgacttcaaggaagatgaggctagttggttatgcgtatgactaattctagtaatcccactacttgaaaatttgaggcaggaggattatgaattccaagccagcctgggatacatagtaagaccaaaagatgaagataaaacagaaacaagctgaaactaaaatgactgaggaggggttagaagagggagttggagtttcctgtcttctgggctgaaggtgtgtctcgcctccattaaccggtgctaggccccggatgtcaccatggcattcctgctagatttttttggtgttaaacattgctctcttcaagagtcataaggacacaggccacccgaagacgtgacatttggtatcaaggaacagtctcagctgtcactgtctctagtgactcagagctagggaatcttgcctagaagtggagggccagcagcaggactggtgaactggtgctgaccaaggtcacactgcctgtggccttctcagctctcctggaggcagggacagactagagaaagtgttatcctgctttgtgctctggtctagccaactggccctgggcttatcccaggttgctgcttagggcaaacgatggccccagcactcagtatatgactagacctacctctactgtctggatggttttttttaaaacactctctggggacgtattacttccttcctggtaggggactgccaagaaagcagcgtggaacttacccctgacagaacagtattgtcccctgggctctgtctaggatgttccttaaaacaatccctcagctgggcctaccctctaagtctttatcctcagacccagaagaggacctgggaggcagccttacagaaatctctagaaatcccttgtctcttctccccagttcaccccaatggctaaattcctgaaactcctgcttctcccccaaaacccaactgaacgtgtcaggtccttcaggatggcgtcacctgctcctagcagttatgtagcccatcactttagccttttgctcacctgcagacgggaagtctcttccacactgtgggaaaacagggtcctgcccaaatcagagcttgcccgaggaatgtggcaaccgctagggagtctgtgccccatgccacttcatgcaggaagcattttgggccagccaatatggctgctttgctggctaatgctgaaagacttttcccccccacattgcctttggtatactattaaaagataagaaaaatgacgagaaggaagaggaagaggaggaagaggaggaagaagaagatgaggaggaggaggaggaggaggaaagatcgatttttttggcctttactttgaaagggctgacatggctgccccacgcccctggtttgagaaaggtttcaacgtgaagcctaggttctgcttattttatgaatctcctgagtgttggggattccagatgagcattcccatgcccaacctgactttatttcagttctgacaatctcctccctgtctctgaacaagattctctgaagatgtcactgactaaccaaccacctagtggattgctccctagagccccatgatgccaggaaaagattcatcctggttggtctgacttgcgttagccagcatgagatcgacagggggaacccccatttctttgggggtacactgtttaaataggttgagagacacagggatctgtttagaatatttcatgtctgccaggcccactacactcccatgtctcctgtcctgcagtgacctgggggagagacgctgaatagaatctattctgggaaatgagcccccaggaagagcaggaatagtgggatacttgtcacttcccctcttctttctagattccctgaggtttcattaagtgacacttactaggtccatatcagtgtacggatgacaccattgtggaatgtgttggaggcaatgtgcagcttgtatggaatccagaagtgacccttcctctgacccagcttcagattgtgtctctaaaatgcatgacggaggtagttggcccatccctaccctgatccgggttactccaaacccttcaggccaagtttcagtctttgcatggctttagtgctttggagagggaaatgtgcagcgtcactgctctggctccctgtacaggctcatctgggaacaccttatacatatatatacacatatactacatgtatatatacatatactatacatatacatatcatggctatctcaggataggaggaggagtctaaagccgtgaagtcgagaagagctgattcccctcaaagtctcttcattttgctccaaacctcagaatcctttactgagtccctcttctgtaggcccgtggcctttcccaagaccacagaactgtggttctcagccttcctaatgctgagaccctttagtacagttcctcatgttgtggtgacccccccagcccccccccacaggcaacataaaaatatttttgttgctacttcataactgtaattttgttactattatggatcataatgtaactctctgacatgcaggccatctgatatgcgacccctgtgaaagggttgttggacagtccctccaaagggttgagggttgagaatcacagctggaggaggtggagatgagaagggcagcgctttgggagccttcagtgtctacagcctctgttatgccacgatgagagacaggggccccctgacagggagctacatttggtgttcttgcggttagagacagacacgtgagacatttggtggctttcctttctgactcctcttagttgcttagagcagggatcaggatcaggtgaggaaacccacaggagggctctggaccagcaaacatttactaactactactttccctgctacaagagagccatccaggaggggactcgggactagtaatgatgggcaggaagggatagcgtgaggagccagctcccttcctcataagatcctcatactactgctccattggaattggggtgcccaagaggtgccagcacccacccagcttcagtactgtgtgcagagggatggagatagtggattacactgggggggggggcaataggaagattgttgagttctctttctcagaagtccagcagatctgggtgagggctgggactggttggtccctctcttcccacaggtatcggagccccctcttgttcccaggggagcctaggagcagctgggccaaagcccaaccaggaatttttccaggctggttcctatatccaagggtgggttagaggtgggggttttggagagctcttaaggaagacactgaggacatggagaaggggacttagtagaaagagaggggacagagccacacgggctaagtgtgcaagcatagagaaatagccaagggttttagggagaatgggaggacagagtacacccctgaattctgtttagaagatgaaccgtaagcctaggctagaactgagggagcctctactcccacccttccgagggttggcggcagcgctgggcagctggcctacctacctttctgaatgctagggtaggtttgaatcaccatgccggcctggcccgcttctgcccccattggcaccctggcttcagttccctggcaacatctctgtgtgtgtgtgtgtgtgtgtgtgagagagagagatcaggaggaacaagggcctctgtctgcccagctgtctctccttcagggctctgccagactacacagtgcatacgtgggtttccacaggtcgtctcactccccgccactgactaactccagaactccacttcccgttctcagtgccacaaatttggtgccaaattctctccagagaagcctctctggaaacttcccagaggatcccattcaccccagggccctagctcctgatgactgcagatcagacaagggctcagataagcatactcccccccccccgtaaccccctccccacatataaacctagagttatgcttccgaggtcaaacacgcaactttttgggtgtgtgtgtatgtcagaaacacgcaattatttgggagctcaaagtctgccgcactcaagaatcaactctcaccccctttccaagacccgtgccatttgagcaagagttggggtgtgcataatgtagtcactagggggcgctcggccatcacggggagatcgtgaacttgggcgagccgagtctgcgtgagggaggacgcgtgtgtcaatgtgagtgcgtgcatgcatgtgtgtgtgtgtgtagtgtgtttgtgaggtgggggagaaagccaggggtcactctagttgtccctatcctcatacgtccctgccagctctccgccttccaacccctactttctcctatattcctgggaaagggaattgttcttagaccctgtccgcatataacctcactctcctgtctcccctgattcccaatactctgggattcccagtgtgttcctgagcccagtttgaaggggtgcacagataattttgaggccgtggaccctggtaaggggtttagctttccatttcgcggtagtggcctaggggctccccgaaaggcggtgcctggctccaccagaccgctccccggggcgggtctgggcggggcttgggggtggagctagatttcctctttttcttccaccgctgttaccggtgagaa

SEQ ID NO: 2 - DNA, 5001 bp, Homo sapiens
  human VEGFA promoter nucleotide sequence
tggctcacgcctgtaatcccagcactctgggaggcagaggtgggccgatcacttgaggtcaggagttcgagaccagcctgggcaacatggtgaaacaccatctctactaaaaacacaaaaattagccaggtgtggtggcaggcacctgcagtcccagctactccggaggctgaggcaggagaattgctcgaacctgggaggcaggggttgcagtgagccgacatggcgccactgcactccagtctgggcgacagagtgagaccctatctcaaaaaaaaaaaaaaaaaaaaaagacccaactcaagtatcatctccaggaagccttcccctactcccagcaattaaatgctcctcagagaattcccatttttggtttactctttggtttacctccagacaggaagcccccactgacactgttgtagtcccagggtgcaacacaaagcagagatcacaagctgagtttaataattgcttgtggaatacatgtcccaagccacctcctgcaggaagcccttccagatgcccattctagccagtctggctctttgcttccataccttcacaacacttgtgcctcccccagggcctctttctcatcttgctttctggggcagctgtgtgcacatttgtctgtgtgcagcaactctctaaggcagggatttttactcctatttttgatgaggggagctgtggctcagagaggttgaataacctaaggccacacagtgagtggcagagccaggaatgtgacttgggtccatttgaatccaaagtccctgtactttccactgccctacctagatgtccctgtacctcctataaaatcagcatggagcctggtgcctggtagtccctacaaatattcacaaattggagcttagctcagctctcaggcaaggcccaggtcaaaagggcagatacagctttgggaccttagttgccaccacatgccataccttcttcccagcagaaggactccctccaagacagggtaggggtggaggatgtgaacaggggcagaaatgggcatgttttggggtcagacttggaggaatagcagagattggagtgtcagaaggtgagcatgcctgggggtgttggggagatgcaattcatcagggacagcttagtgtcaggggattagactggggcccatgaaggagaggcagaggctgatgggcctaggggtggtgtgggtaggtgagcttccccagacagtgactctgccctgccctctctccagctaggtcctcttccccattccttccccctttcctgactggatcctcttgggagagttaccctccttggcttcctctgctccaatctttttatcagttggccatcattacttatcattacctcaagtcaaacctccagatccacatggggctaggacattggcactggaccaaagaggccctttcctttgctttctctttgtctttttaatgctttgttgcaaagacctaggcggggagagagagagagagagagagacagagattgacccacagtcagggtcagggaattgaggggaaccaacccaattctctctccttcaattcaccaggtttgtatcctgcccttcctgcagatcagtgtcctgctagtcacctgggggtcaggggatggagtgaaggacaagacctccttccattgcagtgaagccacttggagaaatgtgtggaaaacagcaagacccagtgactctctcctcaccttctttccaatctcaggagagattttgtcccttcatccaccggcttctagattaaccacccacacccacacaggcgagagtttccctgaatattggaggtgacaggacatcaggacaaagtacaactattgtgccttggcccaatcactactttctttgtctggggccgccgctggctcctggctgcctttcgcacttttctccacccccacccctttctccccttccccctcacctggaacaccttccccttcctccttggctccttctgaactgccttcagagccacagactgtggggagtggccactgcgctcccaaggtgaggccctccaagcggggccgagtttgcccctcaactgggagccagcatgacctctgtgtgggctgctctctgcttcactgccccttcccccaatctgctaggtgaccctgggcccctttgtgccctctctgggccttcggaggattctttggggagacagtctgctctgacgccccttcccctgcagcaagcagcctggggagggaggtgaggataagtgaagtcaagttgttcagggggctaagcccatggaagggaagatgccacagagatacatgtggtcttgtgattgttgttttgtgctttttccccttttttgaaagctcaggtgactaggtgacttgagcttttaatttggtgacaatgtgggcactggctgagtccttaagagtacattgttgtaaatgccggtgacaacacactggggcatgggatccagagttaacccctccaggtcacagccaggttatatctccacaatgaaggggggaggtgggccatactttctcgccctaatgaaggtagctcaaaaacccctaggccaggttgtaatcctagccttatataaaaggaattctgtgccctcactcccctggatccctgggcaaagccccagagggaaacacaaacaggttgttgtaacacaccttgctgggtaccaccatggaggacagttggcttatgggggtggggggtgcctggggccacggagtgactggtgatggctatccctccttggaacccctccagcctcctcttagcttcagatttgtttatttgttttttactaagacctgctctttcaggtctgttggctcttttaggggctgaagaaggccgagttgagaagggatgcaagggagggggccagaatgagcccttagggctcagagcctccatcctgccccaagatgtctacagcttgtgctcctggggtgctagaggcgcacaaggaggaaagttagtggcttcccttccatatcccgttcatcagcctagagcatggagcccaggtgaggaggcctgcctgggagggggccctgagccaggaaataaacatttactaactgtacaaagaccttgtccctgctgctggggagcctgccaagtggtggagacaggactagtgcacgaatgatggaaagggagggttggggtgggtgggagccagcccttttcctcataagggccttaggacaccataccgatggaactgggggtactggggaggtaacctagcacctccaccaaaccacagcaacatgtgctgaggatggggctgactaggtaagctccctggagcgttttggttaaattgagggaaattgctgcattcccattctcagtccatgcctccacagaggctatgccagctgtaggccagaccctggcaagatctgggtggataatcagactgactggtcccactcttcccacaggcctcagagccccaactttgttccctggggcagcctggaaatagccaggtcagaaaccagctaggaatttttccaagctgcttcctatatgcaagaatgggatggggcctttgggagcacttagggaagatgtggagagttggaggaaaagggggcttggaggtaagggaggggactgggggaaggataggggagaagctgtgagcctggagaagtagccaagggatcctgagggaatgggggagctgagacgaaacccccatttctattcagaagatgagctatgagtctgggcttgggctgatagaagccttggcccctggcctggtgggagctctgggcagctggcctacagacgttccttagtgctggcgggtaggtttgaatcatcacgcaggccctggcctccacccgcccccaccagccccctggcctcagttccctggcaacatctggggttgggggggcagcaggaacaagggcctctgtctgcccagctgcctccccctttgggttttgccagactccacagtgcatacgtgggctccaacaggtcctcttccctcccagtcactgactaaccccggaaccacacagcttcccgttctcagctccacaaacttggtgccaaattcttctcccctgggaagcatccctggacacttcccaaaggaccccagtcactccagcctgttggctgccgctcactttgatgtctgcaggccagatgagggctccagatggcacattgtcagagggacacactgtggcccctgtgcccagccctgggctctctgtacatgaagcaactccagtcccaaatatgtagctgtttgggaggtcagaaatagggggtccaggagcaaactccccccaccccctttccaaagcccattccctctttagccagagccggggtgtgcagacggcagtcactagggggcgctcggccaccacagggaagctgggtgaatggagcgagcagcgtcttcgagagtgaggacgtgtgtgtctgtgtgggtgagtgagtgtgtgcgtgtggggttgagggcgttggagcggggagaaggccaggggtcactccaggattccaatagatctgtgtgtccctctccccacccgtccctgtccggctctccgccttcccctgcccccttcaatattcctagcaaagagggaacggctctcaggccctgtccgcacgtaacctcactttcctgctccctcctcgccaatgccccgcgggcgcgtgtctctggacagagtttccgggggcggatgggtaattttcaggctgtgaaccttggtgggggtcgagcttccccttcattgcggcgggctgcgggccaggcttcactgagcgtccgcagagcccgggcccgagccgcgtgtggaagggctgaggctcgcctgtccccgccccccggggcgggccgggggcggggtcccggcggggcggagccatgcgccccccccttttttttttaaaagtcggctggtagcggggaggat

SEQ ID NO: 3 - RNA, 24 nt, Mus musculus
  miR-466c-5p (MIMAT0004877)
ugaugugugugugcauguacauau

SEQ ID NO: 4 - RNA, 22 nt, Mus musculus
  miR-466c-3p (MIMAT0004878)
auacauacacgcacacauaaga

SEQ ID NO: 5 - RNA, 24 nt, Mus musculus
  Synthetic RNA comprising custom GU-repeat throughout the sequence. Repeat-miR-466c-5p
ugauguguguguguguguguguau

SEQ ID NO: 6 - RNA, 23 nt, Mus musculus
  Synthetic RNA that has GU-repeat containing seed combined with scrambled sequence with no existing RNA target (custom fusion). Fusion-miR466c-5p
ugauguguguguaugmgagauac

SEQ ID NO: 7 - DNA, 19 bp, Mus musculus
  Forward cloning oligo of miR-466c for lentiviral vector
agagtcaggaagatcagga

SEQ ID NO: 8 - DNA, 19 bp, Mus musculus
  Reverse cloning oligo of miR-466c for lentiviral vector
ctagcaagcattttcactc

SEQ ID NO: 9 - DNA, 23 bp, Mus musculus
  M-miR-466 1a miR-466 1 gRNA T
gatccgcatatacacatacatag

SEQ ID NO: 10 - DNA, 23 bp, Mus musculus
  M-miR-466 1b miR-466 1 gRNA B
aaacctatgtatgtgtatatgcg

SEQ ID NO: 11 - DNA, 23 bp, Mus musculus
  M-miR-466 2a miR-466 2 gRNA T
gatccgtgagcacacagacacag

SEQ ID NO: 12 - DNA, 23 bp, Mus musculus
  M-miR-466 2b miR-466 2 gRNA B
aaacctgtgtctgtgtgctcacg

SEQ ID NO: 13 - DNA, 21 bp, Mus musculus
  466del forward primer for CRISPR deletion detection PCR
tcaggagtgcaagttcatggt

SEQ ID NO: 14 - DNA, 21 bp, Mus musculus
  466del reverse primer for CRISPR deletion detection PCR
ggattgatgagtgccattccc

SEQ ID NO: 15 - RNA, 21 nt, Homo sapiens
  hsa-miR-297 sequence (MIMAT0004450)
auguaugugugcaugugcaug

SEQ ID NO: 16 - RNA, 22 nt, Homo sapiens
  hsa-miR-574-3p
cacgcucaugcacacacccaca

SEQ ID NO: 17 - RNA, 23 nt, Mus musculus
  Repeat only miR-466c
uguguguguguguguguguguau

SEQ ID NO: 18 - RNA, 24 nt, Mus musculus
  Seed repeat miR-466c-5p
uguguguggugugcauguacauau

SEQ ID NO: 19 - RNA, 24 nt, Mus musculus
  Seed-fusion miR-466c-5p
uaaggcuagugugcauguacauau

SEQ ID NO: 20 - DNA, 28 bp, Homo sapiens
  hCD9 forward primer
gccgccaccatgccggtcaaaggaggca

SEQ ID NO: 21 - DNA, 19 bp, Artificial Sequence
  hCD9 reverse primer
gaccatctcgcggttcctg

SEQ ID NO: 22 - DNA, 30 bp, Artificial Sequence
  hCD9 forward primer
agggggatccaccgggccgccaccatgccg

SEQ ID NO: 23 - DNA, 27 bp, Artificial Sequence
  hCD9 reverse primer
gcggcggcgaccatctcgcggttcctg

SEQ ID NO: 24 - DNA, 22 bp, Artificial Sequence
  Forward primer for HA.FLAG.hAgo2
gatggtcgccgccgccatggac

SEQ ID NO: 25 - DNA, 37 bp, Artificial Sequence
  Reverse primer for HA.FLAG.hAgo2
gaggttgattgtcgatcaagcaaagtacatggtgcgc

SEQ ID NO: 26 - DNA, 27 bp, Artificial Sequence
  hTSG101 forward primer
gccgccaccatggcggtgtcggagagc

SEQ ID NO: 27 - DNA, 20 bp, Artificial Sequence
  hTSG101 reverse primer
gtagaggtcactgagaccgg

SEQ ID NO: 28 - DNA, 33 bp, Artificial Sequence
  hTSG101 forward primer
ccccagggggatccaccgggccgccaccatggc

SEQ ID NO: 29 - DNA, 31 bp, Artificial Sequence
  hTSG101 reverse primer
tggcggcggcgtagaggtcactgagaccggc

SEQ ID NO: 30 - DNA, 22 bp, Artificial Sequence
  Forward primer for HA.FLAG.hAgo2
tgacctctacgccgccgccatg

SEQ ID NO: 31 - DNA, 42 bp, Artificial Sequence
  Reverse primer for HA.FLAG.hAgo2
atccagaggttgattgtcgatcaagcaaagtacatggtgcgc

SEQ ID NO: 32 - DNA, 3159 bp, Homo sapiens
  LV.CD9.Ago2 cDNA
agggggatccaccggatgccggtcaaaggaggcaccaagtgcatcaaatacctgctgttcggatttaacttcatcttctggcttgccgggattgctgtccttgccattggactatggctccgattcgactctcagaccaagagcatcttcgagcaagaaactaataataataattccagcttctacacaggagtctatattctgatcggagccggcgccctcatgatgctggtgggcttcctgggctgctgcggggctgtgcaggagtcccagtgcatgctgggactgttcttcggcttcctcttggtgatattcgccattgaaatagctgcggccatctggggatattcccacaaggatgaggtgattaaggaagtccaggagttttacaaggacacctacaacaagctgaaaaccaaggatgagccccagcgggaaacgctgaaagccatccactatgcgttgaactgctgtggtttggctgggggcgtggaacagtttatctcagacatctgccccaagaaggacgtactcgaaaccttcaccgtgaagtcctgtcctgatgccatcaaagaggtcttcgacaataaattccacatcatcggcgcagtgggcatcggcattgccgtggtcatgatatttggcatgatcttcagtatgatcttgtgctgtgctatccgcaggaaccgcgagatggtctagatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgcgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagacgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgacggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttgatcgacaatcaacctc

SEQ ID NO: 33 - DNA, 3645 bp, Homo sapiens
  hTSG101.Ago2 cDNA
agggggatccaccggatggcggtgtcggagagccagctcaagaaaatggtgtccaagtacaaatacagagacctaactgtacgtgaaactgtcaatgttattactctatacaaagatctcaaacctgttttggattcatatgtttttaacgatggcagttccagggaactaatgaacctcactggaacaatccctgtgccttatagaggtaatacatacaatattccaatatgcctatggctactggacacatacccatataatccccctatctgttttgttaagcctactagttcaatgactattaaaacaggaaagcatgttgatgcaaatgggaagatatatcttccttatctacatgaatggaaacacccacagtcagacttgttggggcttattcaggtcatgattgtggtatttggagatgaacctccagtcttctctcgtcctatttcggcatcctatccgccataccaggcaacggggccaccaaatacttcctacatgccaggcatgccaggtggaatctctccatacccatccggataccctcccaatcccagtggttacccaggctgtccttacccacctggtggtccatatcctgccacaacaagttctcagtacccttctcagcctcctgtgaccactgttggtcccagtagggatggcacaatcagcgaggacaccatccgagcctctctcatctctgcggtcagtgacaaactgagatggcggatgaaggaggaaatggatcgtgcccaggcagagctcaatgccttgaaacgaacagaagaagacctgaaaaagggtcaccagaaactggaagagatggttacccgtttagatcaagaagtagccgaggttgataaaaacatagaacttttgaaaaagaaggatgaagaactcagttctgctctggaaaaaatggaaaatcagtctgaaaacaatgatatcgatgaagttatcattcccacagctcccttatacaaacagatcctgaatctgtatgcagaagaaaacgctattgaagacactatcttttacttgggagaagccttgagaaggggcgtgatagacctggatgtcttcctgaagcatgtacgtcttctgtcccgtaaacagttccagctgagggcactaatgcaaaaagcaagaaagactgccggtctcagtgacctctactgaatggacatccccaaaattgacatctatcattatgaattggatatcaagccagagaagtgcccgaggagagttaacagggaaatcgtggaacacatggtccagcactttaaaacacagatctttggggatcggaagcccgtgtttgacggcaggaagaatctatacacagccatgccccttccgattgggagggacaaggtggagctggaggtcacgctgccaggagaaggcaaggatcgcatcttcaaggtgtccatcaagtgggtgtcctgcgtgagcttgcaggcgttacacgatgcactttcagggcggctgcccagcgtcccttttgagacgatccaggccctggacgtggtcatgaggcacttgccatccatgaggtacacccccgtgggccgctccttcttcaccgcgtccgaaggctgctctaaccctcttggcgggggccgagaagtgtggtttggcttccatcagtccgtccggccttctctctggaaaatgatgctgaatattgatgtgtcagcaacagcgttttacaaggcacagccagtaatcgagtttgtttgtgaagttttggattttaaaagtattgaagaacaacaaaaacctctgacagattcccaaagggtaaagtttaccaaagaaattaaaggtctaaaggtggagataacgcactgtgggcagatgaagaggaagtaccgcgtctgcaatgtgacccggcggcccgccagtcaccaaacattcccgctgcagcaggagagcgggcagacggtggagtgcacggtggcccagtatttcaaggacaggcacaagttggttctgcgctacccccacctcccatgtttacaagtcggacaggagcagaaacacacctaccttcccctggaggtctgtaacattgtggcaggacaaagatgtattaaaaaattaacggacaatcagacctcaaccatgatcagagcgactgctaggtcggcgcccgatcggcaagaagagattagcaaattgatgcgaagtgcaagtttcaacacagatccatacgtccgtgaatttggaatcatggtcaaagatgagatgacagacgtgactgggcgggtgctgcagccgccctccatcctctacgggggcaggaataaagctattgcgacccctgtccagggcgtctgggacatgcggaacaagcagttccacacgggcatcgagatcaaggtgtgggccattgcgtgcttcgccccccagcgccagtgcacggaagtccatctgaagtccttcacagagcagctcagaaagatctcgagagacgccggcatgcccatccagggccagccgtgcttctgcaaatacgcgcagggggcggacagcgtggagcccatgttccggcacctgaagaacacgtatgcgggcctgcagctggtggtggtcatcctgcccggcaagacgcccgtgtacgccgaggtcaagcgcgtgggagacacggtgctggggatggccacgcagtgcgtgcagatgaagaacgtgcagaggaccacgccacagaccctgtccaacctctgcctgaagatcaacgtcaagctgggaggcgtgaacaacatcctgctgccccagggcaggccgccggtgttccagcagcccgtcatctttctgggagcagacgtcactcacccccccgccggggatgggaagaagccctccattgccgccgtggtgggcagcatggacgcccaccccaatcgctactgcgccaccgtgcgcgtgcagcagcaccggcaggagatcatacaagacctggccgccatggtccgcgagctcctcatccagttctacaagtccacgcgcttcaagcccacccgcatcatcttctaccgcgacggtgtctctgaaggccagttccagcaggttctccaccacgagttgctggccatccgtgaggcctgtatcaagctagaaaaagactaccagcccgggatcaccttcatcgtggtgcagaagaggcaccacacccggctcttctgcactgacaagaacgagcgggttgggaaaagtggaaacattccagcaggcacgactgtggacacgaaaatcacccaccccaccgagttcgacttctacctgtgtagtcacgctggcatccaggggacaagcaggccttcgcactatcacgtcctctgggacgacaatcgtttctcctctgatgagctgcagatcctaacctaccagctgtgtcacacctacgtgcgctgcacacgctccgtgtccatcccagcgccagcatactacgctcacctggtggccttccgggccaggtaccacctggtggataaggaacatgacagtgctgaaggaagccatacctctgggcagagtaacgggcgagaccaccaagcactggccaaggcggtccaggttcaccaagacactctgcgcaccatgtactttgcttgatcgacaatcaacctc